(19) United States
(12) Patent Application Publication
(10) Pub. No.: US 2009/0324536 A1
(43) Pub. Date: Dec. 31, 2009

(54) ANTIMICROBIAL POLYMERS AND COATINGS

(75) Inventors: Yuyu Sun, Sioux Falls, SD (US); Zhengbing Cao, Sioux Falls, SD (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(73) Assignee: THE UNIVERSITY OF SOUTH DAKOTA, Vermillion, SD (US)

(21) Appl. No.: 12/488,359

(22) Filed: Jun. 19, 2009

Related U.S. Application Data
(60) Provisional application No. 61/145,907, filed on Jan. 20, 2009.
(60) Provisional application No. 61/194,752, filed on Sep. 30, 2008.
(60) Provisional application No. 61/133,164, filed on Jun. 26, 2008.

(51) Int. Cl. A01N 25/10; A01N 43/34; A61K 31/438; B05D 3/02; A01N 43/66; A01P 1/00

(52) U.S. Cl. 424/78.23; 514/278; 514/315; 427/385.5; 424/78.35; 514/245

(57) ABSTRACT

Biocidal compounds have been synthesized and tested. These biocidal compounds have broad-spectrum efficacy and their biocidal properties are easily renewable. Illustrative examples of these biocidal compounds include N-halamine monomers and polymers and silver sulfadiazine polymers. These compounds can be used to add biocidal function to various materials and articles.

### CROSS-REFERENCE TO RELATED APPLICATION

This application claims the benefit under 35 U.S.C. § 119(e) to U.S. Provisional Patent Application No. 61/145,907, filed on Jan. 20, 2009, entitled “POLYMERIC N-HALAMINE LATEX EMULSIONS FOR USE IN ANTIMICROBIAL PAINTS AND COATINGS,” U.S. Provisional Patent Application No. 61/194,752, filed Sep. 30, 2008, entitled “POLYMERIC SILVER SULFADIAZINES AS RECHARGABLE BIOCIDAL POLYMERS: SYNTHESIS AND CHARACTERIZATION,” and U.S. Provisional Patent Application No. 61/133,164, filed on Jun. 26, 2008, entitled “METHOD OF PREPARING AND CHARACTERIZING POLYMERIZABLE HINDERED AMINE-BASED ANTIMICROBIAL MATERIALS,” all of which are incorporated herein by reference in their entirety.

### TECHNICAL FIELD

The invention relates generally to antimicrobial materials and more particularly to renewable or replenishable antimicrobial materials.

### BACKGROUND

Microorganisms have strong abilities to survive on the surfaces of ordinary materials; some species of microorganisms, including drug-resistant strains, can stay alive for more than 90 days. Contaminated materials may serve as significant and important sources for cross-contamination and crossinfection. One of the potential methods to reduce such risks is to introduce antimicrobial properties into materials that are frequently touched and thus potentially have a high risk of spreading disease.

In some cases, a desire to control surface microbial contamination in residential, commercial, institutional, industrial, and hygienic applications has resulted in the development of biocidal polymers. These biocidal polymers are attractive candidates for medical devices, hospital and dental equipment, water purification, food storage and transportation, as well as a broad range of related industrial, environmental, hygienic, and bio-protective applications. In some instances, these polymers can be mixed into other materials and/or can be used to coat existing devices and structures. In some cases, these polymers have been used in antimicrobial paints. While antimicrobial paints and other antimicrobial polymers are commercially available, none of them are believed to provide broad-spectrum function against bacteria, mold, fungi and viruses simultaneously.

### SUMMARY

The invention is directed to renewable antimicrobial compositions and coatings. In some embodiments, the antimicrobial compositions, materials and coatings may be formed from or otherwise include N-halamine materials. In some embodiments, the antimicrobial compositions, materials and coatings may be formed from or otherwise include polymeric sulfadiazine materials.

The following abbreviations are defined as follows:

TMPM is 2,2,6,6-tetramethyl-4-piperidinyl methacrylate.

Cl-TMPM is N-chloro-2,2,6,6-tetramethyl-4-piperidinyl methacrylate.

Poly (Cl-TMPM) is poly(N-chloro-2,2,6,6-tetramethyl-4-piperidinyl acrylate).

TMPMA is 2,2,6,6-tetramethyl-4-piperdyl methacrylate.

PTMPMA refers to polymeric TMPMA or TMPMA grafted onto a substrate.

SD is sulfadiazine.

ASD is acryloyl sulfadiazine.

MMA is methyl methacrylate.

ASD-MMA is a copolymer of ASD and MMA.

C-SD is a class of adducts between cyanuric chloride and sulfadiazine.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a FT-IR spectra of TMPM, Cl-TMPM and Poly(Cl-TMPM).

FIG. 2 illustrates a ¹³C-NMR spectra of TMPM, Cl-TMPM, and Poly(Cl-TMPM).

FIG. 3 illustrates a UV/VIS spectra of TMPM, Cl-TMPM and Poly(Cl-TMPM) in chloroform.

FIG. 4 illustrates DSC curves of TMPM, Cl-TMPM and Poly(Cl-TMPM).

FIGS. 5A, 5B, 5C and 5D are images illustrating paint films of (A) Color Place® exterior latex semi-gloss house paint, white paint, (B) Color Place® exterior latex semi-gloss house paint, white paint containing 20 wt % of poly(Cl-TMPM), (C) Auditions® satin paint, blue paint, and (D) Auditions® satin paint, blue paint containing 20 wt % of poly(Cl-TMPM).

FIGS. 6A and 6B are electronic images illustrating a biofilm-controlling function of the samples against S. aureus [the polymeric N-halamine-containing paint contained 10 wt % of poly(Cl-TMPM)].

FIG. 7 illustrates a positive chlorine content in solution (The polymeric N-halamine-containing paint had 10 wt % of poly(Cl-TMPM), and the total active chlorine content was 1.307%).

FIGS. 8A and 8B are images illustrating a potassium iodine/starch test after 30 sec of contact with (A) a pure commercial paint film, and (B) a paint film containing 5 wt % of poly(Cl-TMPM).

FIG. 9 illustrates the effects of grafting reaction time on graft yield (6.0 g of fabric in 150 ml solution which contained 0.44 mol/L of TMPMA and 3.6 mmol/L of ceric salt at 50-55° C.).

FIG. 10 illustrates the effects of weight ratio of monomer to fabric on graft yield (in 150 ml solution which contained 0.44 mol/L of TMPMA and 3.6 mmol/L of ceric salt at 50-55° C. for 3 hours.).

FIG. 11 illustrates the FT-IR spectra of (a), original cotton fabrics; (b), PTMPMA-grafted-fabrics (graft yield: 17.8%); (c), chlorinated PTMPAM-grafted-fabrics (graft yield: 17.8%) and (d), PTMPMA (prepared in hexane with 0.5% of AIBN as initiator).

FIG. 12 illustrates the TGA curves of (a), original cotton fabric; (b), PTMPMA-grafted-fabric (graft yield: 17.8%); (c), chlorinated PTMPMA-grafted-fabric (graft yield: 17.8%) and (d), pure PTMPMA.

FIG. 13 illustrates the FT-IR spectra of SD, ASD, and ASD-MMA copolymer.

FIG. 14 illustrates the 1H-NMR spectra of SD, ASD, and ASD-MMA copolymer.

FIG. 15 illustrates the XPS spectra of (A) ASD-MMA copolymer, and (B) polymeric silver sulfadiazine (silver content: 1.29%).

FIG. 16 illustrates the TGA curves of (A) ASD-MMA copolymer, and (B) polymeric silver sulfadiazine (silver content: 1.29%).

### DETAILED DESCRIPTION

The invention pertains to antimicrobial materials that can be integrated into or otherwise used with various compositions, materials and coatings to provide the compositions, materials and coatings with long-lasting, renewable and broad-spectrum biocidal activity. In some embodiments, the antimicrobial materials are halogen-bearing compounds such as N-halamines. In other embodiments, the antimicrobial materials are silver-bearing compounds such as polymeric silver sulfadiazine. In some instances, the halogen ions and/or the silver ions, when contacting microbes, are consumed. In some embodiments, the antimicrobial materials are renewable or replenishable, meaning that the halogen or silver ions can be replaced as they are consumed.

### Monomers

An N-halamine is a compound containing one or more nitrogen-halogen covalent bonds. These bonds are formed by the halogenation (such as, for example, chlorination or bromination) of imide, amide, or amine groups. One property of N-halamines is that when microbes come into contact with the N—X structures (X is Cl or Br), a halogen exchange reaction occurs, resulting in the expiration of the microorganisms. The antimicrobial action of N-halamines is believed to be a manifestation of a chemical reaction involving the transfer of positive halogens from the N-halamines to appropriate receptors in the microbial cells. This process can effectively destroy or inhibit the enzymatic or metabolic cell processes, resulting in the expiration of the organisms. Various classes of N-halamine monomers are described herein.

In one embodiment, one or more suitable N-halamines are represented by Formula 1, below:

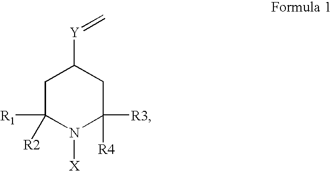

in which R1, R2, R3, R4, and Y can be C₁ to C₄₀ alkyl, C₁ to C₄₀ alkylene, C₁ to C₄₀ alkenyl, C₁ to C₄₀ alkynyl, C₁ to C₄₀ aryl, C₁ to C₃₀ alkoxy, C₁ to C₄₀ alkylcarbonyl, C₁ to C₄₀ alkylcarboxyl, C₁ to C₄₀ amido, C₁ to C₄₀ carboxyl, or combinations thereof, and X can be Cl or Br.

In some embodiments, one or more suitable N-halamine monomers include N-chloro-2,2,6,6-tetramethyl-4-piperidyl methacrylate, N-bromo-2,2,6,6-tetramethyl-4-piperidyl methacrylate, N-chloro-2,2,6,6-tetramethyl-4-piperidyl acrylate, and N-bromo-2,2,6,6-tetramethyl-4-piperidyl acrylate, which are illustrated below as Formulas 2-5, respectively:

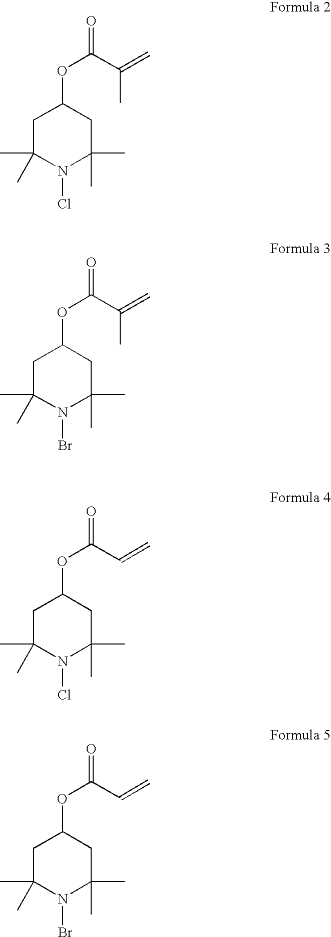

In some embodiments, one or more N-halamine monomers may be represented by Formula 6.

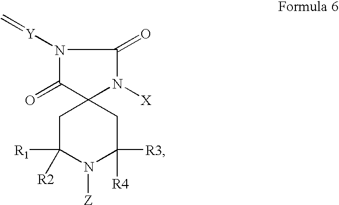

in which R1, R2, R3, R4, and Y are defined as above, X can be Cl, Br or H and Z can be Cl or Br.

In some embodiments, one or more suitable N-halamine monomers are represented by formulas 7-12, respectively, in which X represents Cl, Br or H:

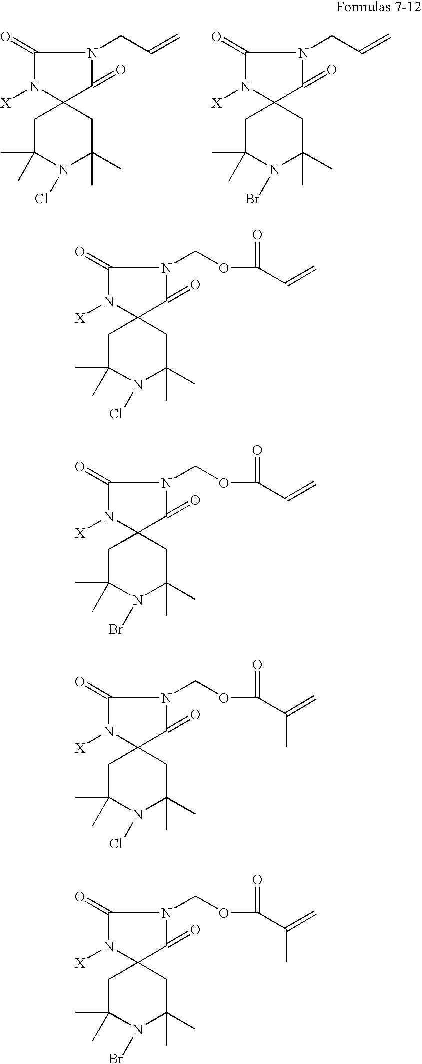

In some embodiments, one or more suitable N-halamine monomers are presented by formulas 13-16, respectively, in which X, Y or Z can each represent Cl, Br or H:

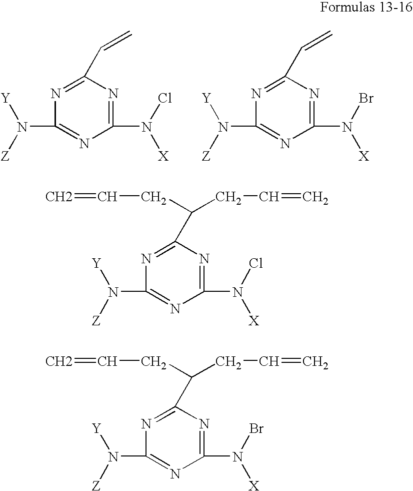

In a particular embodiment, a new polymerizable N-halamine monomer was developed. Cl-TMPM, or N-chloro-2,2,6,6-tetramethyl-4-piperidinyl methacrylate is readily polymerizable using a semi-continuous emulsion polymerization technique, forming stable water-based latex-like emulsions. These polymeric N-halamine latex emulsions can be directly added into commercial water-based latex paints as antimicrobial additives, providing potent antimicrobial activities against bacteria (including the drug-resistant species), mold and other fungi species, and viruses.

### Halogenated Polymers

A new process has been developed for preparing polymeric N-halamines in which a halogenated monomer is polymerized, rather than halogenating after polymerization as is currently done. One of the advantages of the new process is that the monomer is a liquid at room temperature, which means that the monomer can be dispersed evenly into water in the presence of conventional emulsifiers to form stable emulsions, the resulting monomer emulsions could be readily polymerized to form poly(Cl-TMPM) latex emulsions, and the new poly(Cl-TMPM) emulsions could be directly used for antimicrobial applications without the “exposure to a halogen source” step that was needed in the conventional “after-halogenation” polymeric N-halamine preparation approach. In other cases, the pre-halogenated monomer may have different solubility in common solvents from the original unchlorinated monomer, or have other different physical/chemical properties, all of which can be used to alter/modify/improve the process in the formation of halogeneated polymers.

The poly (CL-TMPM) latex emulsions can be directly mixed with commercial water-based latex paints at any ratios without coagulation and/or phase separation. The covering capacity and appearance of the paints are not negatively affected by the presence of the poly(Cl-TMPM) latex emulsions. The new poly(Cl-TMPM)-containing paints provide potent antimicrobial effects against bacteria (including multidrug-resistant species), fungi, and viruses, completely inhibited mold growth, and successfully prevent bacteria biofilm formation on the paint surfaces.

In some embodiments, polymeric N-halamine may be incorporated into a coating or paint to provide an antimicrobial character to the surface of the object on which the coating or paint is applied. In an example, an N-halamine monomer, N-chloro-2,2,6,6-tetramethyl-4-piperidinyl acrylate (Cl-TMPA) was synthesized. Cl-TMPA is a water-insoluble oil-like liquid. Using dioctyl sulfosuccinate sodium as emulsifier and ammonium persulfate [(NH4)2S2O8] as an initiator, Cl-TMPA has been successfully polymerized into poly(N-chloro-2,2,6,6-tetramethyl-4-piperidinyl acrylate), forming latex-like emulsions in water. The polymeric N-halamine latex emulsions act as conventional paints, and they may be painted or sprayed or otherwise conventionally applied onto any solid surfaces (wood, wall, floor, plastic, metal, etc.). On drying, poly (N-chloro-2,2,6,6-tetramethyl-4-piperidinyl acrylate) forms a clear paint film that attaches firmly to solid surfaces.

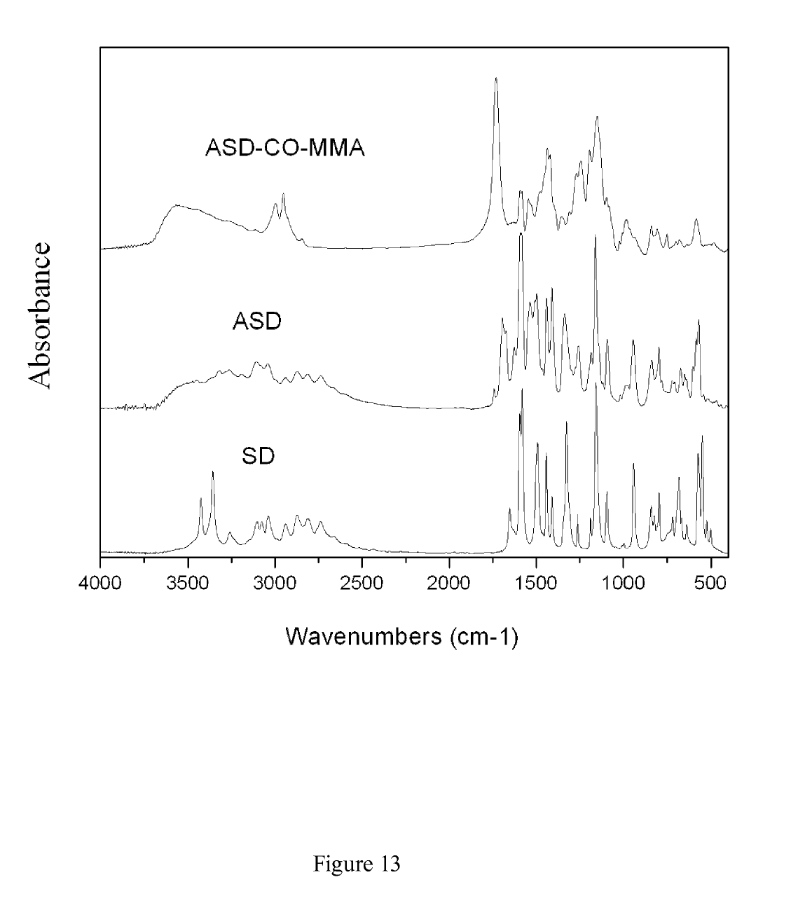

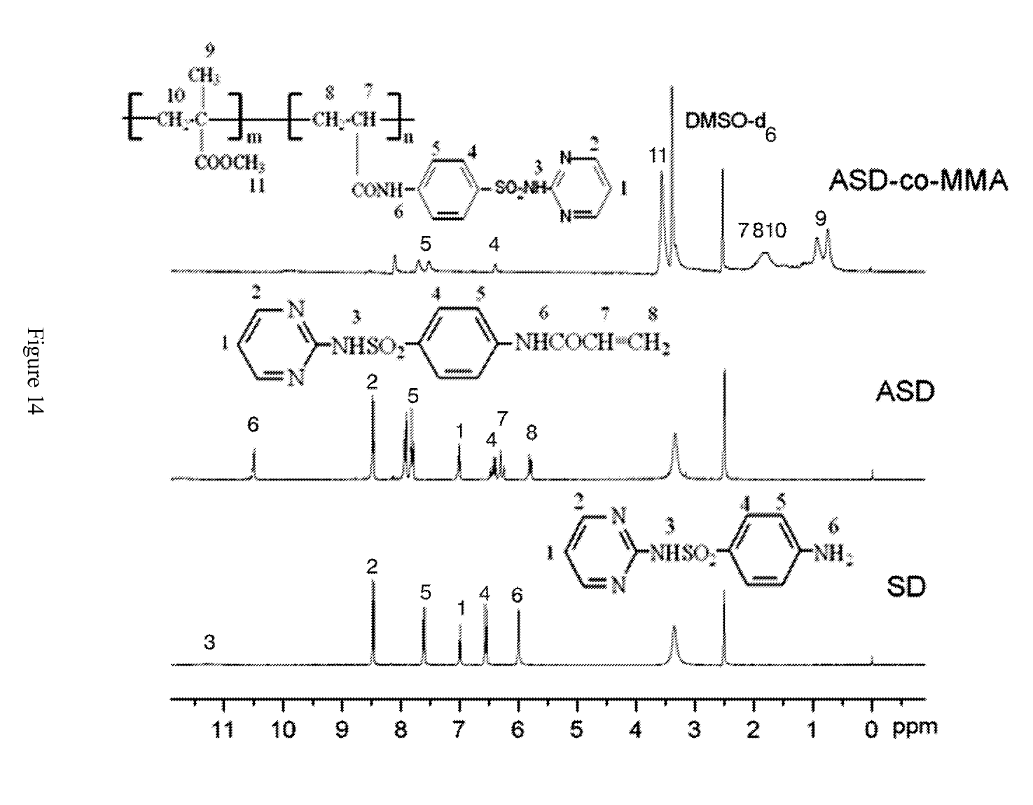

In some embodiments, the polymeric N-halamine latex emulsions may be mixed with water-based coatings or paints to serve as antimicrobial ingredients for the coating or paint. For example, the polymeric N-halamine emulsions may be mixed with a white latex paint (for example, Color Place® latex semi-gloss house white paint) and a blue latex paint (for example, Auditions® satin paint). The N-halamine emulsions were found to freely mix with both paints at any ratio without coagulation and/or phase separation. The film forming capacity of the new paints is similar to those of the original paints. As an example, FIG. 13 shows the same polystyrene plastic films painted with the original paints and the new paint mixtures containing 5% of polymeric N-halamine emulsions.

In some embodiments, the monomers shown in Formulas 2-16 may be homopolymerized or copolymerized with other monomers to form polymers, and the resultant polymers have powerful, durable and rechargeable antimicrobial functions.

The antimicrobial functions have been found to be durable for longer than one year under normal in-use conditions, and can be easily monitored by a potassium iodine/starch test; if challenging conditions (e.g, heavy soil, flooding, etc.) consumed more chlorines and reduced the antimicrobial functions, the lost functions can be readily regenerated by another chlorination treatment. These properties point to great potentials of the new polymeric N-halamines for use in antimicrobial surfacing and/or treatment of a wide range of related residential, commercial, institutional, industrial, and hygienic applications to reduce the risk of microbial contamination.

### Grafting Halogenated Monomers or Polymers

In some embodiments, N-halamine monomers and/or polymers can be grafted onto solid substrates such as fabric. In some cases, this entails a grafting step and a halogenation step. An N-halamine monomer can be grafted (i.e., covalently bonded or ionically bonded) onto any fabric or other substrates having an appropriate binding site. In a particular embodiment, useful N-halamine polymers include poly (N-halo-2,2,6,6,-tetramethyl-4-piperidyl acrylate) and/or poly (N-halo-2,2,6,6-tetramethyl-4-piperidyl methacrylate), as well as copolymers that include poly (N-halo-2,2,6,6,-tetramethyl-4-piperidyl acrylate) and/or poly (N-halo-2,2,6,6-tetramethyl-4-piperidyl methacrylate) segments. In some cases, when grafting onto a polysaccharide-based fabric such as cotton, the ceric ion (Ce4+) redox system may be used as an initiator. Without wishing to be bound by theory, it is believed that Ce4+ may oxidize cellulose, creating free-radical grafting sites primarily at C2 and C3 carbons on the polymer backbones to start the grafting polymerization.

In a particular embodiment, useful N-halamine polymers include poly (N-halo-2,2,6,6-tetramethyl-4-piperidyl acrylate) and/or poly (N-halo-2,2,6,6-tetramethyl-4-piperidyl methacrylate) homopolymers, as well as copolymers that include poly (N-halo-2,2,6,6-tetramethyl-4-piperidyl acrylate) and/or poly (N-halo-2,2,6,6-tetramethyl-4-piperidyl methacryiate) segments. In one example, as will be discussed, a vinyl hindered amine monomer, 2,2,6,6-tetramethyl-4-piperdyl methacrylate (TMPMA), was illustratively grafted onto cotton cellulose. After bleach treatment with diluted sodium hypochlorite solution, the grafted TMPMA moiety was transformed into polymeric amine N-halamines. In another example, Cl-TMPM, or N-chloro-2,2,6,6,-tetramethyl-4-piperidinyl methacrylate was grafted onto solid substrates such as cotton cellulose. All of the grafted substrates provided exceptionally durable and fully renewable antimicrobial activities with good hydrolytic and thermal stabilities.

Poly (N-halo-2,2,6,6-tetramethyl-4-piperidyl acrylate)- and Poly (N-halo-2,2,6,6-tetramethyl-4-piperidyl methaerylate)-based polymeric N-halamines have been demonstrated to be ultra-stable and autoclavable, and provide total kill of gram-negative bacteria, gram-positive bacteria, and fungi in less than 20 minutes. Further, if the chlorine ions are consumed or removed, they can be repeatedly recharged by another bleach treatment. Thus, these new polymers find a wide range of applications, particularly where very stable N-halamines are needed (such as, for example, coatings and paints that are antimicrobial for years without recharging). These polymers also find important applications where autoclave treatment of the products incorporating the antimicrobial character is needed or desired.

### Silver Sulfadiazine Polymers

In some embodiments, polymeric silver sulfadiazines have been found to provide a biocidal compound that exhibits potent, durable, renewable, and non-leaching biocidal activities. Generally, sulfadiazine may be covalently attached to a target polymeric material through chemical reactions between C-SD (cyanuric chloride and sulfadiazine adducts) and reactive site on the material, or free radical homopolymerization or co-polymerization of ASD (acrylol sulfadiazine). Upon exposure to diluted silver nitrate aqueous solutions, the bound sulfadiazine moieties form complexes with silver cations to produce polymeric silver sulfadiazines. The resulting polymeric silver sulfadiazines demonstrate powerful biocidal activities against Gram-negative bacteria, Gram-positive bacteria, and fungi. Extensive use of the polymeric silver sulfadiazines may consume most of the silver cations and reduce the biocidal activities of the polymeric silver sulfadiazines. However, the polymeric silver sulfadiazines may be recharged to replace the consumed or lost silver cations. The recharging of the silver cations may be accomplished, for example, using a silver nitrate treatment to regenerate the biocidal functions.

In some embodiments, C-SD is represented by Formula 17, shown below:

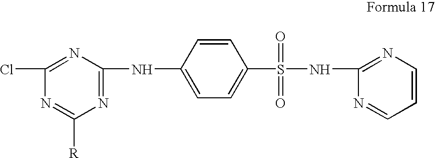

in which R can be C1, C1 to C40 alkyl, C1 to C40 alkylene, C1 to C40 alkenyl, C1 to C40 alkynyl, C1 to C40 aryl, C1 to C30 alkoxy, C1 to C40 alkylcarbonyl, C1 to C40 alkylcarboxyl, C1 to C40 amido, C1 to C40 carboxyl, or combinations thereof.

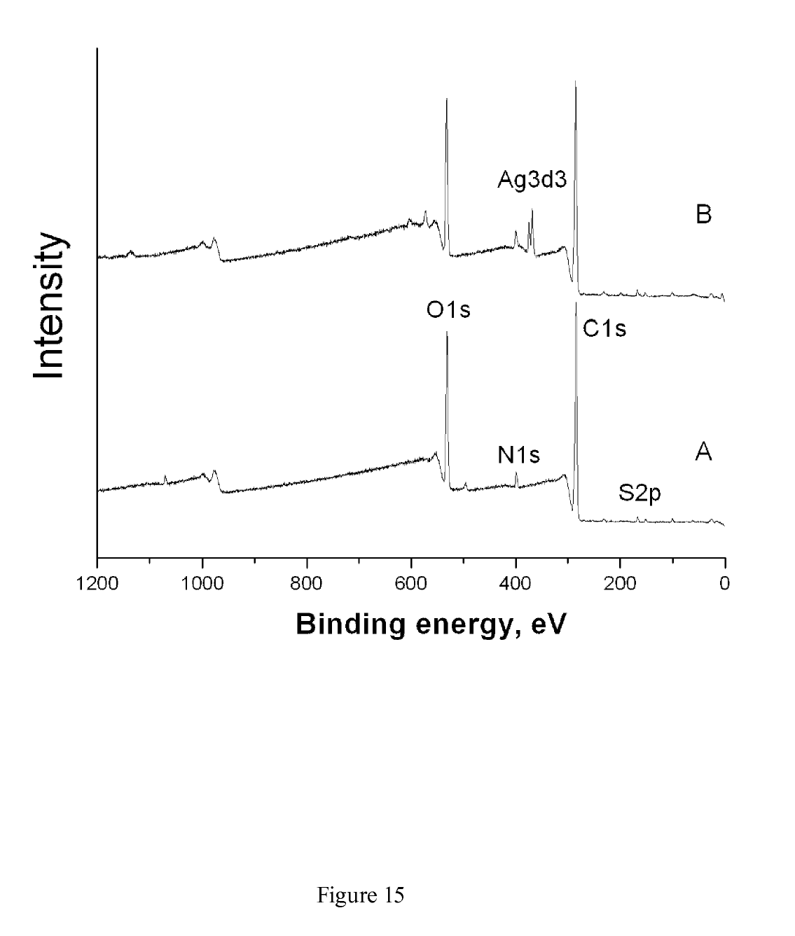

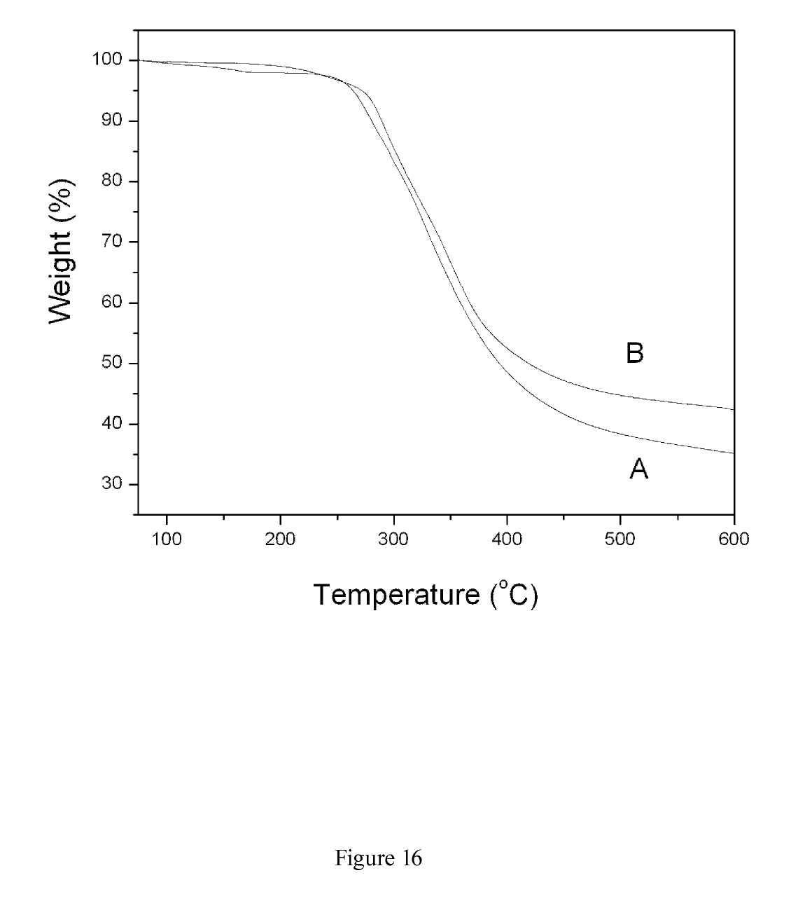

Another embodiment pertains to the preparation of polymeric silver sulfadiazines. Upon exposure to aqueous solutions of silver salts (e.g., silver nitrate), the sulfadiazine moieties in the polymers strongly bind silver cations to form complexes, leading to the formation of polymeric silver sulfadiazines. This transformation was characterized by X-ray photoelectron spectroscopy (XPS) studies, as shown in FIG. 15. In the spectrum of the ASD-MMA copolymers four elements are clearly detected, and they are assigned to oxygen (Ols at 531.8 eV), nitrogen (N₁ₛ at 399.1 eV), carbon (C₁ₛ at 284.6 eV), and sulfur (S₂ₚ at 167.08). After reacting with silver nitrate aqueous solutions, the copolymer was transformed into polymeric silver sulfadiazine. Consequently, in addition to these four elements, a new peak at 374.6 eV can be detected in the XPS spectrum, which is caused by the bound silver (Ag_{3d5}). Quantitative analysis of the XPS data indicates that the surface silver content of the polymeric silver sulfadiazines was 1.29%, which is believed to provide potent biocidal activities against Gram-negative bacteria, Gram-positive bacteria, and fungi (see the discussion below).

### EXPERIMENTAL SECTION

### Materials

Ammonium persulfate [(NH₄)₂S2O], 2,2,6,6-tetramethyl-4-piperidyl methacrylate (TMPM), dichlorisocyanurate sodium (DCCANa), and dioctyl sulfosuccinate sodium (DSS) were purchased from Sigma-Aldrich and used as received. The microorganisms, Staphylococcus aureus (S. aureus, ATCC 6538), Escherichia coli (E. coli, ATCC 15597), Methicillin-resistant S. aureus (MRSA, ATCC BAA-811), Vancomycin-resistant E. faecium (VRE, ATCC 700221), Candida tropicalis (C. tropicalis, ATCC 62690), Stachybotrys chartarum (S. chartarum, ATCC 34915), and MS2 virus (ATCC 15597-B1) were obtained from American Type Culture Collection (ATCC).

The materials employed included cotton fabrics (purchased from Testfabrics Inc.) that were cleaned with acetone to remove impurities before use. 2,2,6,6-tetramethyl-4-piperdyl methacrylate (TMPMA) (Wako chemicals Inc.) was purified by precipitation from acetone solution into water. Escherichia coli (E. coli, ATCC 15597), Staphylococcus epidermidis (S. epidermidis, ATCC 35984) and Staphylococcus aureus (S. aureus, ATCC 6538) were provided by American Type Culture Collection. Cerium (IV) ammonium nitrate (Alfa Aesar), nitric acid (Acros), sodium thiosulfate solution (0.0100 M, Ricca Chemical), potassium iodide (Acros) and other chemicals were analytical grade and used as received.

Sulfadiazine (SD), acryloyl chloride, and silver nitrate were purchased from Aldrich and used as received. 2,2-azobisisobutyronitrile (AIBN, Aldrich) was recrystallized from methanol three times. Methyl methacrylate (MMA, Fisher) was distilled under reduced pressure in the presence of hydroquinone. Dimethyl formamide (DMF, Aldrich) was distilled under vacuum, and dried with 4 A molecular sieves. Other chemicals were analytical grade and used without further purification.

### Instruments

Fourier transform infrared (FT-IR) spectra were recorded on a Thermo Nicolet 6700 FT-IR spectrometer (Woburn, Mass.). ¹³C-NMR studies were carried out using a Varian Unity-200 spectrometer (Palo Alto, Calif.) at ambient temperature in CDCl₃. UV spectra of the samples in chloroform were obtained on a Beckman DU® 520 UV/VIS spectrophotometer. Thermal properties of the samples were characterized using DSC-Q200 (TA instruments, DE) at a heating rate of 10° C./min under N₂ atmosphere. Gel Permeation Chromatography (GPC) studies were performed in THF on a GPC system equipped with a Waters 515 HPLC pump. The dual detection system consisted of a Waters 2414 RI detector and a multiwave length Waters 486 UV detector. The instrument was calibrated using polystyrene standards.

¹H-NMR studies were carried out using a Varian Unity-300 spectrometer (Palo Alto, Calif.) at ambient temperature in DMSO-d₆. X-ray photoelectron spectroscopy (XPS) of the samples were obtained from a PHI 5700 XPS system equipped with dual Mg X-ray source and monochromated Al X-ray source, depth profile and angle resolving capabilities. Thermo Gravimetric Analysis (TGA) was performed on TA Q50 (TA Instruments, DI) under N₂ atmosphere at a heating rate of 10° C./minute. In some cases, thermogravimetric analysis (TGA) was carried out on a TA Q50 Thermogravimetric analyzer at a heating rate of 20° C./min under nitrogen gas (N₂) flow.

### Monomer Preparation

An N-halamine monomer, N-chloro-2,2,6,6-tetramethyl-4-piperidinyl methacrylate (Cl-TMPM), was synthesized through chlorination of 2,2,6,6-tetramethyl-4-piperidinyl methacrylate (TMPM) with DCCANa. In a typical run, a solution of DCCNa (12.1 g, 0.06 mol) in water (50 mL) was added to a solution of TMPM (11.25 g, 0.05 mol) in chloroform (50 mL). The mixture was vigorously stirred at room temperature for 1 h. After filtration, the chloroform layer was separated and dried with magnesium sulphate for 24 h. Magnesium sulphate was filtrated off, and chloroform was evaporated. The residual was recrystallized from water/ethanol at 0° C. Cl-TMPM was obtained as white powders (12.6 g, yield: 96.3%; MP: 15° C. by DSC), and changed to a colorless oil upon storage at room temperature. The production of Cl-TMPM is illustrated below:

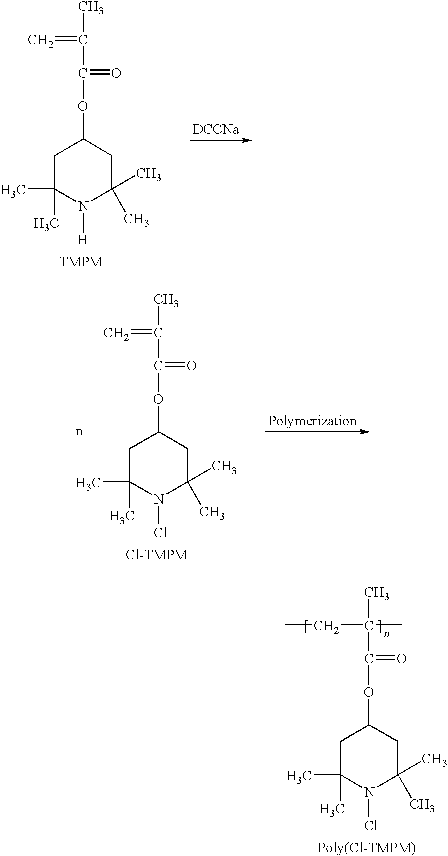

By using similar approaches (the chlorine source can be DCCNa or any other sources that can provide chlorine), the monomers illustrated in Formulas 2-16 were synthesized with good yields.

While TMPM is a solid at room temperature (MP 62° C.), Cl-TMPM has a melting point of 15° C. (by DSC), and it is a clear liquid at room temperature. The liquid nature of Cl-TMPM makes it much easier to disperse Cl-TMPM evenly into water in the presence of conventional emulsifiers to form stable emulsions, which would be difficult to do if TMPM was used. Due to the simplicity in preparation of the monomer and polymer emulsions and the ease in use of the final products, it is highly possible that the pre-chlorination approach can be adopted widely in the preparation of other polymeric N-halamines to control microbial contamination in a broad range of related applications.

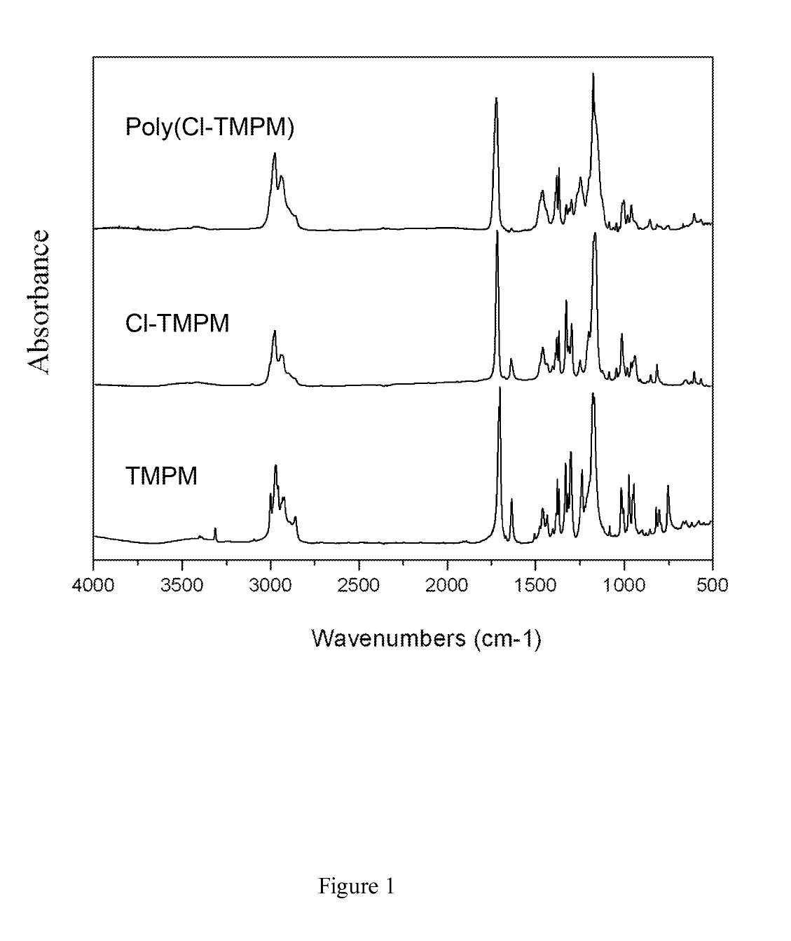

FT-IR analysis was used to follow the reactions. FIG. 1 shows the IR spectra of TMPM, Cl-TMPM, and poly(Cl-TMPM). In the spectrum of TMPM, the 3312 and 3340 cm⁻¹ peaks are attributable to N—H stretching vibrations. The peak at 1635 cm⁻¹ can be related to the carbon-carbon double bonds, and the 1700 cm⁻¹ band was caused by the ester carbonyl, in good agreement with the literature data. Upon chlorination, the N—H structure was transferred into N—Cl. Thus, the N—H stretching vibrations disappeared in the spectrum of Cl-TMPM. Furthermore, the ester carbonyl band shifted from 1700 cm-1 to 1716 cm⁻¹, which could be caused by the breakage of the “C═O—H—N” hydrogen bonds. After polymerization, Cl-TMPM was transformed into poly(Cl-TMPM). As a result, the double bond band around 1635 cm⁻¹ disappeared in the spectrum of poly(Cl-TMPM), and the ester carbonyl band further shifted from 1716 cm⁻¹ to 1721 cm⁻¹.

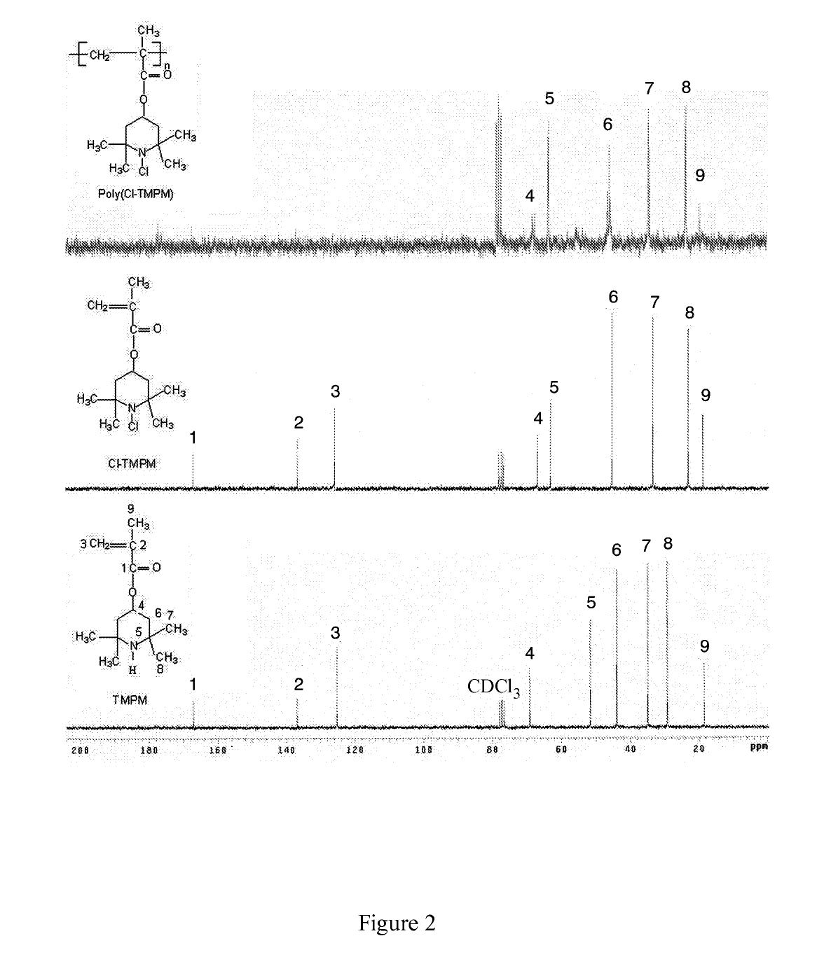

The FT-IR results were confirmed by ¹³C-NMR studies, as shown in FIG. 2. In the spectrum of TMPM, the peaks at 136.8 ppm (C2) and 125.0 ppm (C3) were caused by the carbons of the double bonds, and the signal at 51.5 ppm was related to the two neighboring carbons (C5) of the N—H group. After chlorination, the 51.5 ppm peak shifted to 62.9 ppm in the spectrum of Cl-TMPM. This change was attributed to the replacement of N—H structure with N—Cl group because the latter has stronger electron withdrawing effect than N—H group. After polymerization, the two double bond carbons peaks disappeared in the spectrum of poly(Cl-TMPM), confirming the formation of polymers.

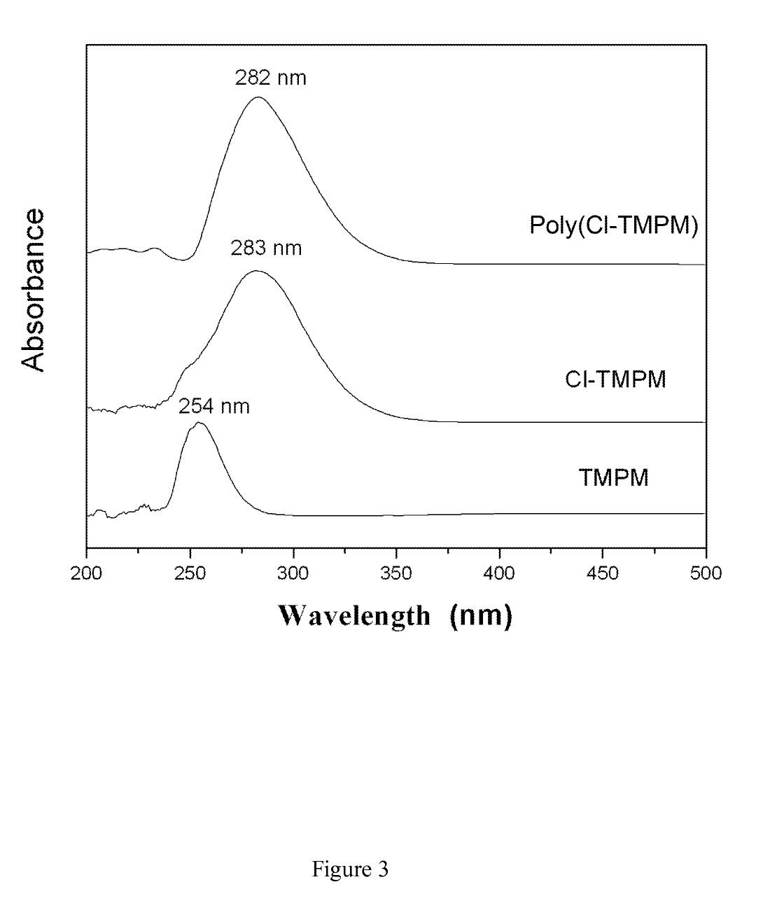

The FT-IR and NMR results agreed well with UV studies. As shown in FIG. 3, TMPM showed an adsorption peak around 254 nm. After chlorination, a strong adsorption peak around 282 nm could be observed in the spectrum of the Cl-TMPM. UV absorptions of N-halamines have been well established, and this peak could be caused by the disruption/disassociating of the N—Cl bond and/or the transition from a bonding to an antibonding orbital, indicating that after chlorination, the —NH groups in TMPM were transformed into —NCl structures. In the spectrum of poly(Cl-TMPM), the N—Cl peak could still be observed, suggesting that the N—Cl structure survived in the emulsion polymerization process. This finding was further strengthened by iodimetric titration, which showed that while Cl-TMPM had 13.68% of active chlorine, after polymerization, the resulting poly(Cl-TMPM) had 13.07% of active chlorine, retaining 95.5% of the theoretical value.

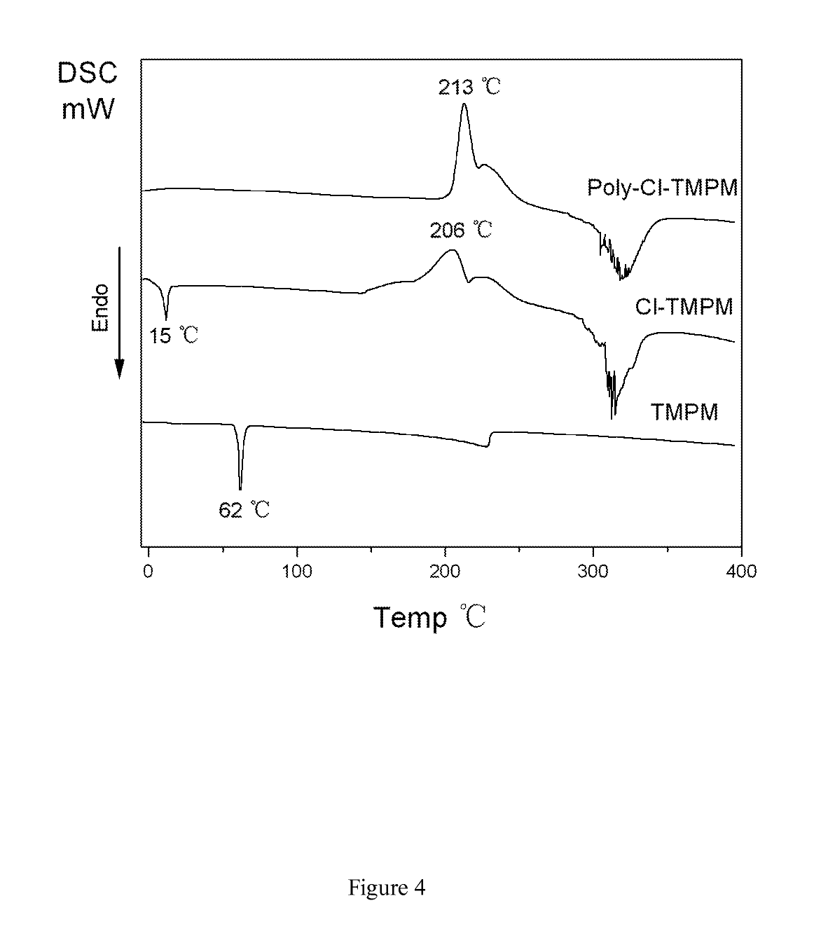

To provide further information about the reactions, the samples were characterized by DSC studies, and the results are presented in FIG. 4. TMPM shows a melting point at 62° C. After chlorination, the N—H bond was transformed into N—Cl bond, and because of the lack of hydrogen bonding, the melting point of Cl-TMPM decreased to 15° C. The broad exothermal peak at 206° C. may be caused by the thermal decomposition of the N—Cl structure. After polymerization, the melting point at 15° C. disappeared, and the N—Cl decomposition temperature slightly increased to 213° C. in the DSC curve of poly(Cl-TMPM). All these findings strongly suggested that Cl-TMPM and poly(Cl-TMPM) latex emulsions have been successfully synthesized following the procedure as illustrated above in Scheme 1. The monomers illustrated in Formulas 2-16 showed similar structural characteristics in FT-IR, NMR, UV-VIS and DSC studies.

### Preparation of Emulsions

Polymerization of Cl-TMPM transformed the monomer into poly(Cl-TMPM) (Mw=5572 Da, and polydispersity=1.94 by GPC), which was a stable water-based emulsion, and could be directly added into commercial latex paints to provide antimicrobial functions. A polymeric N-halamine latex emulsion was prepared by a semi-continuous emulsion polymerization technique as reported previously. Dioctyl sulfosuccinate sodium (DSS) and TX-100 were used as emulsifiers. A stable monomer pre-emulsion was prepared by stirring a mixture of 20% Cl-TMPM, 1% of DDS and 1% of TX-100 in water for 30 min and then sonicating for 10 min. In the first stage of the polymerization, a dispersion of seed particles was prepared by batch emulsion polymerization. In a typical run, the monomer pre-emulsion 1.25 g, water 20 mL, DSS 0.025 g and TX-100 0.025 g were added into a 250 mL three-necked flask equipped with a mechanical stirrer, nitrogen inlet, reflux condenser, and a liquid inlet system. The flask was immersed into a water bath at 70° C. The whole system was thoroughly purged with nitrogen during the reaction. An initiator solution [0.1 g (NH4)₂S₂O₈ in 5 mL water] was added into the reactor. The mixture was stirred for about 30 min until a light blue emulsion appeared.

In the second stage, the monomer pre-emulsion was continuously dropped into the dispersion of the seed particles at a rate of 0.1 mL/min for 3 h. After the addition was completed, the system was further maintained at 70° C. for 0.5 h under constant stirring. The resultant latex emulsion was cooled to room temperature for future use.

To determine the active chlorine contents of the samples, the emulsions were cast into paint films on polytetrafluoroethylene and dried for 1 week at room temperature. Around 0.05 g of the dried paint film was dispersed in 20 mL DMF and 20 mL water containing 1.0 wt % acetic acid. One gram of potassium iodide was added, and the mixture was stirred for 1 h at room temperature under N₂ atmosphere. The released iodine was titrated with 0.01 mol/L sodium thiosulfate aqueous solution. Blank titrations were performed under the same conditions to serve as controls. Percentage of chlorine content was calculated according to the following equation:

$$C l \% = \frac{35.5}{2} \times \frac{( {V}_{C l} - {V}_{0} ) \times {10}^{- 3} \times 0.01}{{W}_{C l}} \times 100 , \qquad ( 1 )$$

where V_{Cl} and V₀ were the volumes (mL) of sodium thiosulfate solutions consumed in the titration of the polymeric N-halamine film and the control, respectively, and W_{Cl} (g) was the weight of the dry film. Each test was repeated three times, and the average was recorded. The monomers illustrated in Formulas 2-16 could be polymerized or co-polymerized in the presence of free-radical initiators to form antimicrobial polymers.

### Preparation of Polymeric N-Halamine-Containing Antimicrobial Paints

The polymeric N-halamine latex emulsions can be directly added into commercial water-based latex paints to provide antimicrobial functions without any phase separation/coagulation. In the current study, a white latex paint (Color Place® latex semi-gloss house white paint, Wal-Mart Stores, Inc, AR) and a blue latex paint (Auditions® satin paint, Valspar Corporation, IL) were used as representative commercial paints. The new paints containing different amounts of polymeric N-halamines were painted onto polystyrene sheets and dried for 7 days at room temperature to prepare paint films.

In an example, an N-halamine monomer, N-chloro-2,2,6,6-tetramethyl-4-piperidinyl acrylate (Cl-TMPA) was synthesized. Cl-TMPA is a water-insoluble oil-like liquid. Using dioctyl sulfosuccinate sodium as emulsifier and ammonium persulfate [(NH4)2S2O8] as an initiator, Cl-TMPA has been successfully polymerized into poly(N-chloro-2,2,6,6-tetramethyl-4-piperidinyl acrylate), forming latex-like emulsions in water. The pathway of this formation is shown below:

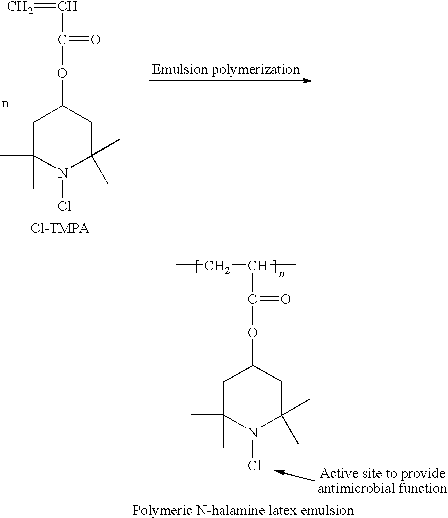

The polymeric N-halamine latex emulsions act as conventional paints, and they may be painted or sprayed or otherwise conventionally applied onto any solid surfaces (wood, wall, floor, plastic, metal, etc.). On drying, poly (N-chloro-2,2,6,6-tetramethyl-4-piperidinyl acrylate) forms a clear paint film that attaches firmly to solid surfaces.

### Preparation of Cl-TMPM and TMPMA-Grafted Fabrics

An amount of TMPMA was dissolved in distilled water containing the equimolar acetic acid to prepare 100 g/L (0.44 mol/L) TMPMA solution, and the final pH value was adjusted to 5-6 with acetic acid. A predetermined amount of cotton fabric was placed in a 250-mL three-necked flask equipped with a condenser and magnetic stirrer. 150 ml of TMPMA solution, 0.30 g (0.55 mmol) of Cerium (IV) ammonium nitrate and 0.5 mL of nitric acid were added into the system. After purging with N₂ for 10 minutes, the reaction system was kept in a water bath (50-55° C.) for 3 hours with constant stirring under a nitrogen atmosphere. Afterwards, the fabrics were washed thoroughly with running hot water, 50% (v/v) of alcohol solution (to remove the homopolymer of TPMPMA which might adhere to the fabric) and distilled water. The fabrics were dried in air overnight and stored in a desiccator to reach constant weights. This process is generally outlined below:

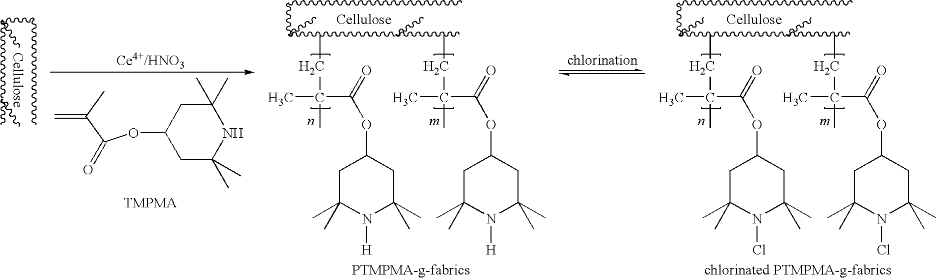

An amount of Cl-TMPM emulsion was prepared using Dioctyl sulfosuccinate sodium (DSS) and TX-100 were as emulsifiers. A predetermined amount of cotton fabric was placed in a 250-mL three-necked flask equipped with a condenser and magnetic stirrer. 150 an amount of Cerium (IV) ammonium nitrate and 0.5 mL of nitric acid were added into the system. After purging with N2 for 10 minutes, the reaction system was kept in a water bath (50-55° C.) for 3 hours with constant stirring under a nitrogen atmosphere. Afterwards, the fabrics were washed thoroughly with running hot water, 50% (v/v) of alcohol solution and distilled water. The fabrics were dried in air overnight and stored in a desiccator to reach constant weights.

In grafting, the ceric ion (Ce⁴⁺) redox system was employed as the initiator. This system has been used as an initiator for grafting vinyl monomers (acrylic acid, acrylamide, acrylonirile, styrene and vinyl acetate, etc.) onto polysaccharides such as starch, cellulose, and chitosan. While not wishing to be bound by theory, it is believed that Ce⁴⁺ may oxidize cellulose, creating free-radical grafting sites primarily at C2 and C3 carbons on the polymer backbones to start the grafting polymerization. In another example, other initiators such as sodium persulfate, benzyl peroxide, etc., also work well in serving as initiators. Also, a pad-dry-cure approach can be used to replace the batch approach to graft Cl-TMPM onto the fabric.

Grafting conditions may influence graft yield. The graft yield was calculated according to equation (1):

$$Graft yield ( \% ) = \frac{( {W}_{g} - {W}_{0} )}{{W}_{0}} \times 100 \qquad ( 1 )$$

where W₀ and W_{g} were the weights of the original and grafted fabrics, respectively. It should be recognized that the above described sequence of events and conditions are merely exemplary of the process, and that the desired result may be accomplished using other steps under other conditions.

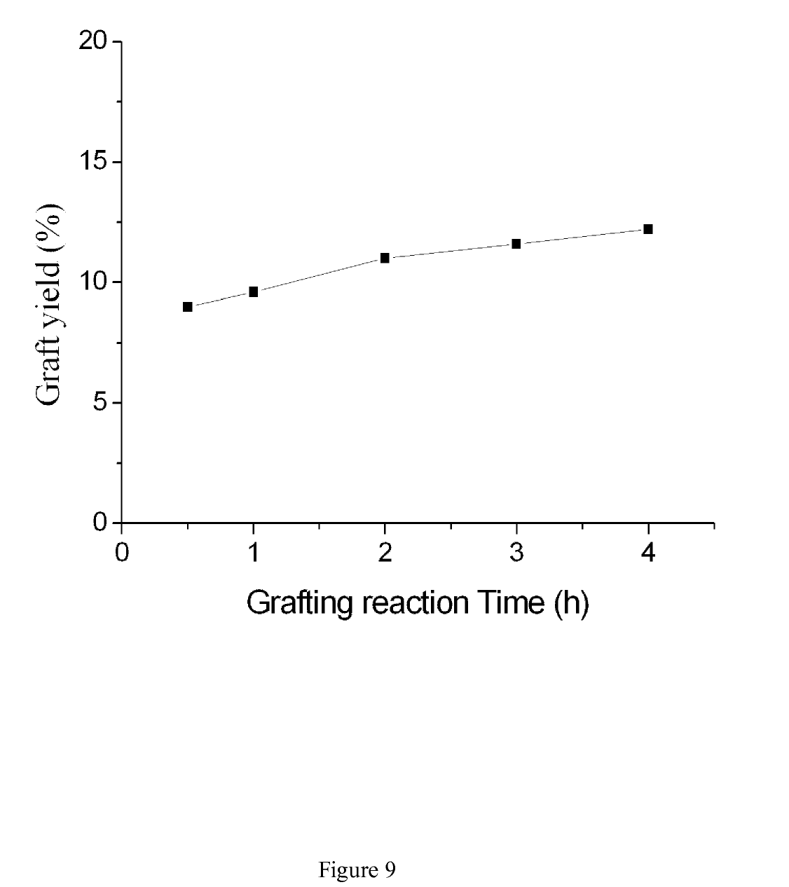

Shown in FIG. 9 are the effects of grafting time on graft yield. It can be seen that the graft yield rapidly increases to 9.0% in the first 30 minutes. After that period of time, the effect of time becomes less obvious: after 3 hours of grafting, the graft yield reaches 11.6%; when the time is further extended to 4 hours, the graft yield slightly increases to 12.2%.

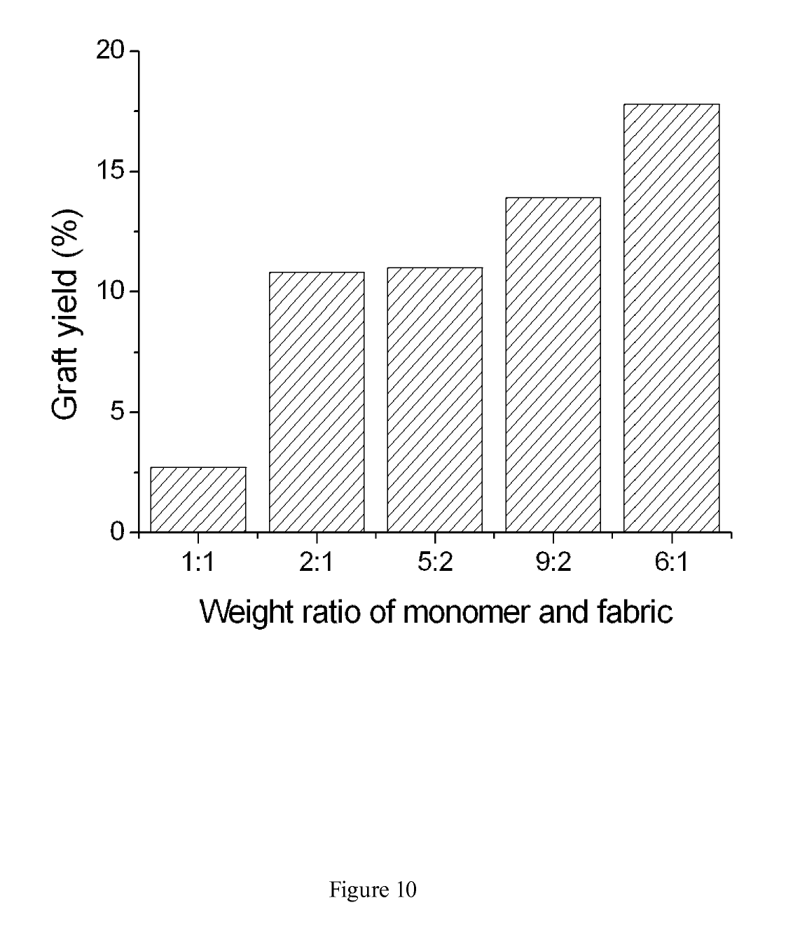

The influences of the weight ratio of TMPMA to the fabric are presented in FIG. 10. Keeping other conditions constant, increasing TMPMA content significantly increases graft yield initially. For example, when the weight ratio of TMPMA to fabric is increased from 1:1 to 2:1, the graft yield markedly increases from 2.7% to 10.8%. In this heterogeneous reaction system, the graft polymerization is believed to largely depend on the diffusion of the monomers into the inner parts of the cotton cellulose. As monomer concentrations go up, more monomers can reach the reactive sites on cotton molecules, leading to higher graft yield. While further increase in TMPMA content could lead to even higher graft yield, at higher than 9/2 weight ratio, gelation of the grafting solution was observed, indicating that too much TMPMA could promote chain transfer reaction to the monomer. Thus, a large amount of TMPMA was consumed in the homopolymerization in the solution, resulting in gel formation.

After the grafting process was performed, the grafted fabric (PTMPMA-grafted-fabric) was chlorinated by diluted sodium hypochlorite aqueous solution. The chlorination of the PTMPMA-grafted-fabrics may then be performed. In an exemplary procedure, the PTMPMA-grafted-fabrics were immersed in 0.1% sodium hypochlorite solution containing 0.05% (v/v) of a nonionic wetting agent (TX-100) under constant stirring for 30 minutes at room temperature. The fabrics were then washed thoroughly with running hot water and distilled water, and dried in air overnight and stored in a desiccator.

During chlorination treatment, the N—H bond of the piperidyl structure in PTMPMA-grafted-fabric was transformed into N—Cl bond, leading to the formation of polymeric amine-based N-halamine structures. Typical results of the chlorination reactions were summarized in the Table below:

ACTIVE CHLORINE CONTENT OF SELECTED
PTMPMA-G-FABRIC

| | Active Chlorine Content |
|---|---|
| Graft Yield (Percent) | (weight percent) |
| 17.8 | 2.56 ± 0.03 |
| 10.8 | 1.55 ± 0.01 |
| 2.7 | 0.45 ± 0.02 |

The active chlorine contents of chlorinated PTMPMA-grafted-fabrics with 17.8%, 10.8% and 2.7% of graft yield are 2.56%, 1.55% and 0.45%, respectively, which are very close to their corresponding theoretical values. Each titration was performed five times. The active chlorine contents of the chlorinated PTMPMA-grafted-fabrics were determined by iodimetric titration with a modified method as reported previously. In the current example, 10˜50 mg of chlorinated PTMPMA-grafted-fabrics were cut into fine powders, and treated with 1 g of KI in 40 mL of 50% ethanol solution (the solution contained 0.05% (v/v) of TX-100 and the pH value was adjusted to 4 with acetic acid) at room temperature under constant stirring for 1 hour. The formed 12 was titrated with standardized sodium thiosulfate aqueous solution. The unchlorinated PTMPMA-grafted fabrics were tested under the same conditions to serve as controls. The available active chlorine content on the fabrics was calculated according to equation (2):

$$Cl \% = \frac{35.5}{2} \times \frac{( {V}_{S} - {V}_{0} ) \times {C}_{{Na}_{2} {S}_{2} {O}_{3}}}{{W}_{S}} \times 100 \qquad ( 2 )$$

where V_{S}, V₀, C_{NaS2O3} and W_{S} were the volumes (mL) of sodium thiosulfate solutions consumed in the titration of the chlorinated and unchlorinated samples, the concentration (mol/L) of the standardized sodium thiosulfate solution, and the weight of the chlorinated sample (mg), respectively. Again, it should be recognized that the above described sequence of events and conditions are merely exemplary of the process, and that the desired result may be accomplished using other steps under other conditions.

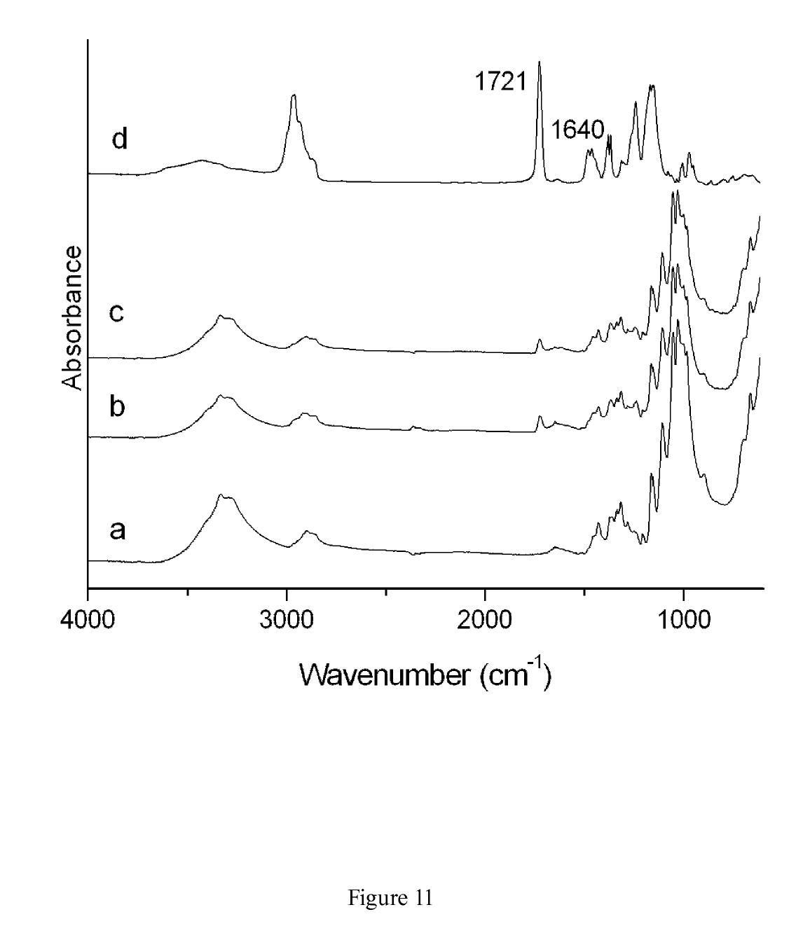

The grafting and chlorination reactions were followed with FT-IR studies. FIG. 11 shows the FT-IR spectra of the original fabric, the PTMPAMA-grafted-fabric before and after chlorination, and the homopolymer of TMPMA (PTMPMA, prepared in hexane with 0.5% of AIBN as initiator at 70° C. for 3 hours). In the spectrum of the original cotton fabric (FIG. 11a), the broad peak above 3000 cm⁻¹ is assigned to the hydroxyl group, and the weak band at 1640 cm⁻¹ is caused by water of hydration. After grafting, a new peak at 1721 cm⁻¹ can be observed in the spectrum of the PTMPMA-grafted-fabric (FIG. 11b). This peak is attributable to stretching vibration of the ester carbonyl groups of the grafted PTMPMA chains, which is confirmed by the spectrum of pure PTMPMA (FIG. 11d), suggesting that PTMPMA has been successfully grafted onto the cotton fabric. After chlorination, the N—H bond of the piperidine structure in PTMPAM-grafted-fabric was transformed into N—Cl bond. Unfortunately, due to the rather weak IR absorbance of N—Cl bond and the relatively low content of PTMPMA in the fabric, little difference could be detected between the spectra of the unchlorinated and chlorinated PTMPMA-grafted-fabrics (FIGS. 11b and 11c).

In other examples, TMPMA was replaced by Cl-TMPM (N-chloro-2,2,6,6-tetramethyl-4-piperidinyl methacrylate) and/or other monomers disclosed as Formulas 1-16, and the grafting reaction also occurred in the presence of suitable initiators (such as Ce⁴⁺, sodium persulfate, benzyl peroxide, and the like) in either a batch process or a pad-dry-cure process.

### Preparation of Silver Sulfadiazine-Based Materials

As illustrated below, in one example, the preparation of silver sulfadiazine-based polymeric biocides may include three basic steps, including synthesizing acryloyl sulfadiazine (ASD), copolymerizing ASD with methyl methacrylate (MMA), and binding silver cations onto the ASD-MMA copolymers. The resultant polymeric silver sulfadiazines demonstrate potent, durable, and rechargeable biocidal functions against Gram-negative bacteria, Gram-positive bacteria, and fungi.

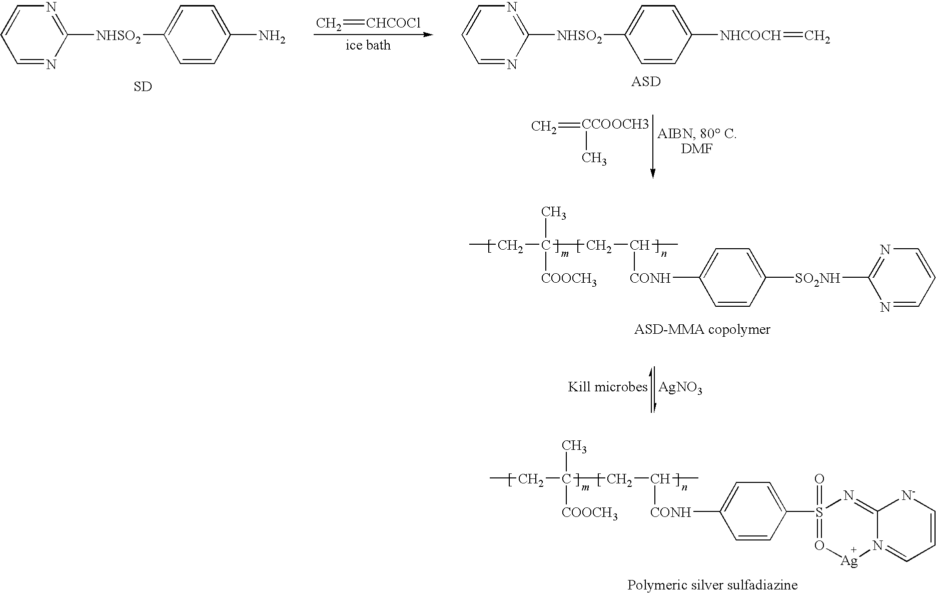

Acryloyl sulfadiazine (ASD) was synthesized according to a method reported previously. Briefly, 0.02 mol of sulfadiazine was dissolved in 60 mL of dry DMF in the presence of 0.022 mol of NaHCO₃ and 5 mg hydroquinone. The mixture was cooled to 0° C., and a solution containing 0.022 mol acryloyl chloride in 20 mL of dry DMF was slowly dropped into the system. After stirring at 0° C. for 6 hours, the whole system was slowly warmed up to room temperature and reacted overnight. After filtration, the solvent was distilled off under reduced pressure, and the resultant viscous residue was washed twice with deionized water. The isolated product was recrystallized twice from methanol, and dried over CaCl₂ in a vacuum oven to obtain 3.80 g yellowish powders (Yield: 62.5% based on SD).

Copolymers of ASD and MMA were synthesized in dry DMF using AIBN as an initiator. In each run, known amounts of ASD, MMA, and AIBN (5 mol % of the monomers) were dissolved in a certain amount of dry DMF in a 3-neck round flask. The reaction was carried out under N₂ atmosphere with constant stirring at 70° C. for 4 hours. At the end of the reaction, the solution was poured into copious 0.2 M NaOH aqueous solutions. The precipitated copolymers were filtered, washed with deionized water, and purified 3 times by repeatedly dissolving in DMF and precipitating from 0.2 M NaOH solutions. After washing with deionized water to neutral pH, the copolymers were filtered out and dried in a vacuum oven at 50° C. for 72 hours to reach constant weights.

In an initial step of synthesizing ASD and ASD-MMA copolymers, ASD was obtained as yellowish crystalline powders through the nucleophilic substitution reaction of sulfadiazine (SD) with acryloyl chloride. ASD has a melting point of 168° C. (measured by DSC), and was easily dissolved in DMF, dimethyl sulfoxide (DMSO), and diluted base solutions.

The acrylic functionality provides ASD with reactive sites to form homopolymers and copolymers through free-radical polymerizations. One significant function of the system of the disclosure is to covalently attach a small amount of ASD moieties into conventional polymers so as to form complexes with silver cations to achieve antimicrobial functions, and thus the copolymerization of ASD with commercially important monomers such as MMA is a significant advantage. Experimentation showed that ASD copolymerized smoothly with MMA in dry dimethyl formamide (DMF) using 2,2-azobisisobutyronitrile (AIBN) as an initiator. A broad range of ASD/MMA monomer molar ratios (from 9/95 to 50/50) were evaluated in the screening studies, and a 10/90 ASD/MMA monomer molar ratio in copolymerization was selected for further investigations, as this was the lowest ASD content to bind sufficient silver cations to provide a total kill of approximately 10⁸ to 10⁹ CFU/mL of bacteria and fungi within 30 minutes without affecting the film-forming capabilities of the samples, as discussed below.

Fourier transform infrared (FT-IR) analysis was used to characterize the reactions. In the spectrum of SD, the 3422, 3355, and 3258 cm⁻¹ peaks are attributable to N—H stretching vibrations, the 1652 and 1580 cm⁻¹ bands are caused by the phenyl and pyrimidinyl rings, and the 1352 and 1157 cm⁻¹ peaks are assigned to γ(SO₂)_{asymmetric} and γ(SO₂)_{symmetric}, respectively, which agrees with literature data. In the spectrum of ASD, the C═O stretching vibration of the acryloyl groups presents at 1694 cm⁻¹. A weak band at 1626 cm⁻¹ can also be observed, which can be related to the carbon-carbon double bonds. After copolymerization with MMA, in addition to the characteristic ASD bands (e.g., a broad peak at 3566 cm⁻¹ for N—H stretching, and peaks at 1591 and 1557 cm⁻¹ for phenyl and pyrimidinyl rings), an intensive peak at 1732 cm⁻¹ is apparent, and this peak is assigned to the carbonyl groups in the ester bonds of the MMA moieties in the copolymers.

The FT-IR results were confirmed by ¹H-NMR studies. In the spectrum of SD, the phenyl amino protons showed a peak at 6.0 ppm, and the sulfonamide proton displayed a weak peak at 11.3 ppm; the signals in the range of 6.5-8.8 ppm correspond to the hydrogen atoms on the phenyl and pyrimidinyl rings. After reacting with acryloyl chloride, SD is transformed into ASD. Thus, the 6.0 ppm peak disappears, and a new peak at 10.5 ppm appears in the ¹H-NMR spectrum of ASD, which is caused by the proton of the newly formed amide groups. In addition, two new peaks at 6.3 ppm (m, 1H, —CH═CH₂) and 5.8 ppm (m, 2H, —CH═CH₂) relating to protons on the acrylic double bonds can also be observed, further confirming the chemical structure of ASD. The spectrum of the ASD-MMA copolymer not only displays signals in the range of 6.5-8.8 ppm (protons on the phenyl and pyrimidinyl rings) that are caused by the polymerized ASD moieties, but also shows resonances at 3.6 ppm (H¹¹) and in the range of 0.7-0.9 ppm (H⁹) that are related to the polymerized MMA structures. Moreover, no peaks corresponding to protons on unsaturated acrylic moieties can be detected, confirming the purity of the copolymer samples.

Transparent ASD-MMA copolymer films (thickness: 0.1-0.2 mm) were obtained using a Carver Heated Press (Model: 3912) at 200° C., 6000 PSI, for 5 minutes. The resultant films were immersed in 0.01 M silver nitrate (AgNO₃) aqueous solutions at room temperature for 24 hours to form polymeric silver sulfadiazine complexes. After silver binding, the films were washed copiously with deionized water (the washing water was tested with potassium iodide to ensure that no further unbound silver cations could be washed off from the samples), air-dried, and stored in a desiccator before use.

In another example, polymeric silver sulfadiazine was prepared by reacting C-SD with a polymer having suitable reactive sites (such as —OH, —NH₂, —SH and the like), as illustrated below. R is as defined previously.

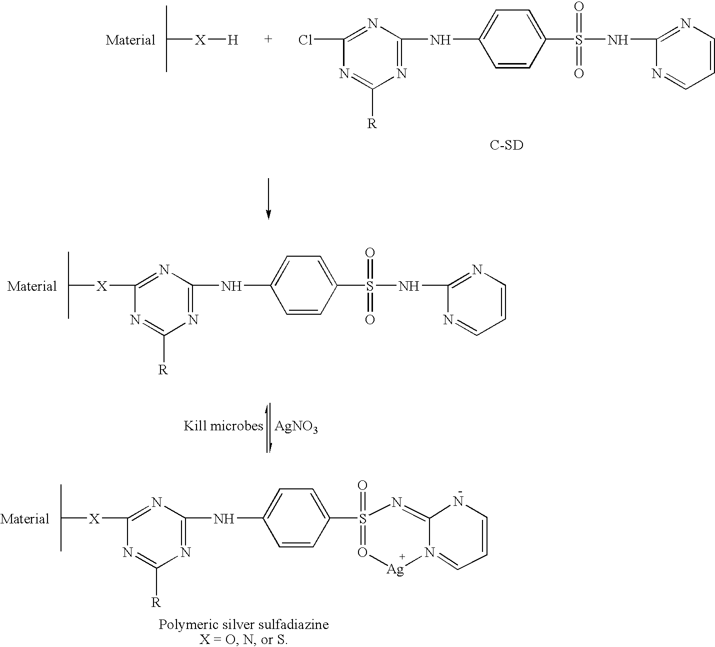

### Antimicrobial Testing Procedures

All microbial tests were performed in a Biosafety Level 2 hood. The guidelines provided by the U.S. Department of Health and Human Services were followed, and appropriate protective equipment including gowns and gloves and recommended decontamination protocols were used to ensure lab safety. In the antibacterial study, Staphylococcus aureus (S. aureus, ATCC 6538) and Escherichia coli (E. coli, ATCC 15597) were used as typical examples of non-resistant Gram-positive and Gram-negative bacteria, respectively. Methicillin-resistant S. aureus (MRSA, ATCC BAA-811) and Vancomycin-resistant E. faecium (VRE, ATCC 700221) were selected to represent drug-resistant strains because these species have caused serious healthcare-associated infections (HAIs) and community-acquired infections. Candida tropicalis (C. tropicalis 62690) was employed to challenge the antifungal activities of the samples, and E. coli bacteriophage MS2 15597-B1 virus was used to represent viral species.

To prepare the bacteria or yeast suspensions, S. aureus 6538, E. coli 15597, MRSA BAA-811, and VRE 700221 were grown in the corresponding broth solutions (see Table 1) at 37° C. for 24 h, and C. tropicalis 62690 was grown in YM broth at 26° C. for 36 h.

| | | | Drug-resistant | | | | |
|---|---|---|---|---|---|---|---|
| | Bacteria | | bacteria | | Yeast | Virus | Mold |
| | S. aureus | E. coli | MRSR | VRE | C. tropicalis | MS2 | S. chartarum |
| | 6538^{a} | 15597^{b} | BAA-811^{a} | 700221^{a} | 62690 | 15597-B1 | 34915 |
| | | | | | | | spore |
| Broth | Tryptic^{c} | LB | Tryptic | Tryptic | YM broth^{d} | EC Medium | N/A |
| | soy | broth^{d} | soy broth | soy | | broth^{c} | |
| | broth | | | broth | | | |
| Agar | Tryptic^{d} | LB | Tryptic | Tryptic | YPD agar^{d} | LB agar^{c} | Cornmeal |
| | soy agar | agar^{c} | soy agar | soy | | | agar^{d} |
| | | | | agar | | | |

^{a}Gram-positive bacteria;
^{b}Gram-negative bacteria;
^{c}Purchased from Difco Laboratories (Detroit, MI);
^{d}Purchased from Fisher Scientific (Fair Lawn, NJ).

Cells were harvested by centrifuge, washed twice with sterile phosphate buffered saline (PBS), and then re-suspended in sterile PBS to 10⁸-10⁹ CFU/mL. In the preparation of the viral suspensions, the freeze-dried bacteriophage MS2 virus was dispersed into Difco™ EC Medium broth containing 10⁸-10⁹ CFU/mL of 24 h-old E. coli 15597 as hosts. The viral suspension was diluted with EC Medium broth to 10⁸-10⁹ plaque forming units (PFU)/mL.

### Testing Polymeric Paints

A modified AATCC (American Association of Textile Chemists and Colorists) Test Method 100-1999 was used to evaluate the antimicrobial efficacies of the polymeric N-halamine-containing paint films. In this test, 200 μL of a bacterial, yeast, or viral suspension were placed onto the surface of a polymeric N-halamine-containing paint film (ca. 2×2 cm), and the film was then “sandwiched” using another identical film to ensure full contact. After different periods of contact time, the entire “sandwich” was transferred into 10 mL of sterilized sodium thiosulfate (Na₂S₂O₃) aqueous solution (0.03 wt %). The mixtures were vigorously votexed for 1 min and sonicated for 5 min to separate the films, quench the active chlorines, and detach adherent cells from the film surfaces into the solution. The resultant solutions were serially diluted, and 100 μl of each diluent were placed onto the corresponding agar plates (see Table 1). In the testing of MS2 virus, the diluent was placed onto LB agar plate overlaid with LB soft agar containing 24 h-old E. coli 15597 as host, as suggested by ATCC. The same procedure was also applied to the original commercial paint films to serve as controls. Viable microbial colonies (for bacteria and yeast) or lysis (for MS2 virus) on the corresponding agar plates were visually counted after incubation at 37° C. for 24 h (in the testing of the bacterial and viral species) or at 26° C. for 36 h (in the testing of C. tropicalis 62690). Each test was repeated three times, and the longest minimum contact time for a total kill of the microbes (the weakest antimicrobial efficacy observed) was reported. This test was designed to simulate possible microbial challenges in real applications when microorganisms were suspended in water.

The antimicrobial activity of the polymeric N-halamine-containing paint films under airborne conditions was evaluated according to a method reported previously. This method was designed to evaluate the antimicrobial activity of the paint against microorganisms that were in air or from coughing/sneezing of infected humans/animals. In the current study, S. aureus 6538, E. coli 15597, MRSA BAA-811, VRE 700221 and C. tropicalis 62690 were grown and harvested as described above. For each bacteria or yeast strain, 200 μL of a microbial suspension (108-109 CFU/mL) were sprayed onto a paint film (4×4 cm) in a biosafety hood using a commercial sprayer. After a certain period of contact time (10-60 min), the film was transferred into 10 mL of sterilized sodium thiosulfate solution (0.03%). After votexing and sonication, the solution was serially diluted, and 100 μL of each diluent were placed onto the corresponding agar plates (See Table 1). Viable microbial colonies on the agar plates were visually counted after incubation at 37° C. for 24 h (for the bacteria) or at 26° C. for 36 h (for the yeast), as described above. Each test was repeated 3 times, and the longest minimum contact time for a total kill of the microbes (the weakest antimicrobial efficacy observed) was reported. The original commercial paint films were evaluated under the same conditions as controls.

The anti-mold efficacy of the new polymeric N-halamine-containing paints was tested with spores derived from Stachybotrys chartarum (S. chartarum, ATCC 34915). S. chartarum is a toxin-producing species that commonly found in buildings with significant water damages, and it is responsible for mold growth. S. chartarum was cultured on cornmeal agar plates at 37° C. until a profusion of conidia was present. Once this was achieved, the culture plate was washed using 10 mL of sterile PBS and 0.1% Tween 80 solution to separate the conidia from the spore. Spore concentration was determined through serial dilution, plating, and enumeration, and the final concentration for the anti-mold test was adjusted to 108-109 CFU/mL with sterile PBS.

In each test, 200 μL of the mold solution was inoculated onto the surface of a polymeric N-halamine-containing paint film (ca. 4×4 cm). The film was placed in a sterile Petri dish containing 1 mL of sterile water. The dish was closed and placed into a static microbial test chamber (ca. 32×39×51 cm) constructed following ASTM D6329-98 (2008). The chamber was sealed and the internal condition was maintained at 100% RH and room temperature. Growth of S. chartarum on the films was inspected weekly within a 3-month test period, and mold growth at each inspection was recorded by measuring the covering ratios of visible mold on the film surfaces. Triplicate sample films were processed for each paint formulations (the original commercial paint, and the new paints containing different amounts of polymeric N-halamines).

The ability of the polymeric N-halamine-containing paint film to prevent biofilm formation was evaluated using SEM analysis. In this study, S. aureus 6538 was grown and harvested as described above. A polymeric N-halamine-containing paint film (ca. 1×1 cm) was immersed in 10 mL sterile PBS containing 10⁸-10⁹ CFU/mL of the bacteria. The mixture was gently shaken at 37° C. for 30 min. The film was taken out of the bacteria solution and gently washed 3 times with 10 mL sterile PBS to remove loosely attached bacteria. The film was immersed into tryptic soy broth and incubated at 37° C. for 3 days. After incubation, the film was rinsed gently with 0.1 M sodium cacodylate buffer (SCB), and fixed with 3% glutaraldehyde in SCB at 4° C. for 24 h. After being gently washed with SCB, the samples were dehydrated through an alcohol gradient method, and dried in a critical point drier. Thereafter, the samples were mounted onto sample holders, sputter coated with gold-palladium, and observed under a Hitachi S-3200N scanning electron microscope. The same procedure was also applied to the original commercial paint films to serve as controls.

In a zone of inhibition test, the surface of a tryptic soy agar plate and Luria-Bertant (LB) agar plate were overlaid with 1 mL of 10⁸-10⁹ CFU/mL of S. aureus 6538 and E. coli 15597, respectively. The plates were then allowed to stand at 37° C. for 2 h. Each polymeric N-halamine-containing paint film (1×1 cm) was placed onto the surface of each of the bacteria-containing agar plates. The film was gently pressed with a sterile forceps to ensure full contact between the film and the agar. The same procedure was also applied to the original commercial paint film to serve as controls. After incubation at 37° C. for 24 h, the inhibition zone around the films was measured. Afterwards, the films were removed sterilely from the agar plates, and washed gently with non-flowing sterile PBS (3×10 mL) to remove loosely attached bacteria. The resultant films were vortexed for 1 min and sonicated for 5 min in 10 mL PBS to detach adherent bacteria. The solution was serially diluted, and 100 μL of each dilution was plated onto the corresponding agar plates (see Table 1). Recoverable microbial colonies were counted after incubation at 37° C. for 24 h.

To investigate the stability of the chorines in the N-halamines, a series of polymeric N-halamine-containing paint films (ca. 2×2 cm) were immersed in 10 mL deionized water under constant shaking (50 rpm) at room temperature. After a certain period of time, 1 mL solution was taken out of the immersing water and tested with a Beckman DU® 520 UV/VIS spectrophotometer in the range of 190-400 nm to determine whether TMPM or Cl-TMPM-containing compounds were released from the paint film into the solution (characteristic absorption peaks of pure TMPM: 254, and Cl-TMPM: 285 nm). Afterwards, the water sample was iodometrically titrated to determine the level active chlorines in the soaking solutions.

The polymeric N-halamine-containing paint films were tested for retention of antimicrobial functions under storage. Paint films with known chloride contents were stored under normal lab conditions (25° C., 30-90% RH). The chloride contents and the antibacterial and antifungal functions were tested periodically over a 12-month storage period.

To test rechargeability, the polymeric N-halamine-containing paint films were first treated with 0.1 M sodium thiosulfate aqueous solution at room temperature for 24 h to quench the bound chloride, and then wiped using a cellulosic cleaning cloth with 1 wt % of DCCNa aqueous solution for 30 sec. The films were left to air-dry overnight, washed with distilled water to remove the remaining DCCNa and air dried. After different cycles of this “quenching-recharging” treatment, the chloride contents and antibacterial and antifungal functions of the resultant films were reevaluated.

### Testing Cl-TMPM and PTMPMA-Grafted Fabrics

The antibacterial properties of the Cl-TMPM and PTMPMA-grafted fabrics were conducted according to a modification of AATCC Test Method 100-1999. In the testing, S. aureus, S. epidermidis and E. coli were grown in broth solutions (tryptic soy broth for S. aureus and S. epidermidis, and Luria-Bertant, or LB broth, for E. coli) for 24 hours at 37° C. The bacteria were harvested with a centrifuge, washed with phosphate-buffered saline (PBS), and then resuspended in PBS to densities of 10⁶-10⁷ CFU/mL. The freshly prepared bacterial suspensions (100 μL) were placed on the surfaces of four square-swatches of the chlorinated PTMPMA grafted cotton cellulose (1 inch×1 inch per swatch). After a certain period of contact time, the swatches were transferred into 10 mL of sterilized sodium thiosulfate solution (0.03%), sonificated for 5 minutes, and vortexed for 60 seconds. The solution was serially diluted, and 100 μL of each diluent were placed on agar plates (LB agar for E. coli and tryptic soy agar for S. aureus and S. epidermidis). The colony-forming units on the agar plates were counted after incubation at 37° C. for 24 hours. Pure cotton fabric and the correspondent unchlorinated PTMPAM grafted cotton fabrics were tested under the same conditions to serve as controls. Each test was repeated three times.

Durability of the antimicrobial properties was tested with machine washing following AATCC Test Method 124-2001. AATCC standard reference detergent 124 was used in all the machine-washing tests.

To test the rechargeability of the active chlorines, the Cl-TMPM grafted fabrics and the chlorinated PTMPMA-grafted-fabrics were first treated with 0.3% of sodium thiosulfate solution for 1 hour to partially quench the active chlorine, and then rechlorinated with the same conditions in the preparation of the first generation of the N-halamine fibrous materials. After a number of cycles of this “bleaching-quenching-bleaching” treatment, the chlorine content and antimicrobial functions of the samples were retested.

### Testing Silver Sulfadiazine Materials

The thermal properties of the silver sulfadiazine samples were evaluated by Thermo Gravimetric Analysis (TGA) analysis. In the range of 75-600° C., the weight loss of the polymeric silver sulfadiazine is 58.5%, and that of the ASD-MMA copolymer is 65.5%. These results suggest that the formation of silver(I)-sulfadiazine coordination complexes stabilizes the polymer structures (see FIG. 15), leading to less weight loss upon heating.

Considering the antibacterial and antifungal activities of the products, in the antimicrobial tests, E. coli, S. aureus, and C. tropicalis were used as representative examples of Gram-negative bacteria, Gram-positive bacteria, and fungi, respectively. Both pure poly (methyl methacrylate (PMMA) and ASD-MMA copolymer (without silver nitrate treatments) films were used as controls.

In parallel to the antibacterial and antifungal studies conducted, a series of the polymeric silver sulfadiazine films (2×2 cm) were immersed in 100 mL deionized water under constant shaking at room temperature, and an UV/VIS spectrophotometer was used to test the immersing solutions. Within the test period of 24 hours, in the range of approximately 190 to approximately 400 nm, no UV absorption was detected. Moreover, potassium iodine test did not show any color change of the immersing solutions. These results suggest that no detectable monomeric SD/ASD components or silver cations were released into the surrounding environment under the testing conditions, indicating that the polymeric silver sulfadiazines might provide biocidal functions primarily through direct contact.

A zone of inhibition test was performed to provide further information about any “contact kill” mechanism of action, and showed that neither pure PMMA and ASD-MMA copolymer nor the polymeric silver sulfadiazine films provided any inhibiting zone during the test period of 24 hours. After zone of inhibition test, the film samples were washed and sonicated to recover surface adherent bacteria.

The antibacterial activity of the polymeric silver sulfadiazines was evaluated according to AATCC (American Association of Textile Chemists and Colorists) Test Method 100 against Staphylococcus aureus (S. aureus, ATCC 6538) and Escherichia coli (E. coli, ATCC 15597) in a Biosafety Level-2 hood. The polymeric silver sulfadiazine films were cut into small pieces (ca. 2×2 cm). Approximately 10 μL of an aqueous suspensions containing 10⁸-10⁹ CFU/mL of S. aureus or E. coli were placed onto the surface of a film. The film was then “sandwiched” using another identical film, and a sterile weight (100 g) was added onto the films. After a certain period of contact time, the entire “sandwich” was transferred into 10 mL sterile PBS. The mixture was sonicated for 5 minutes and vigorously vortexed for 1 minute to separate the films and transform the adherent cells into PBS. An aliquot of the solution was serially diluted, and 100 μL of each dilution were plated onto agar plates (tryptic soy agar for S. aureus, and Luria-Bertant agar for E. coli). The same procedure was also applied to pure poly methyl methacrylate (PMMA) films and the correspondent ASD-MMA copolymer films (without silver nitrate treatment) to serve as controls. Bacterial colonies were counted after incubation at 37° C. for 24 hours. Each test was repeated three times.

In the experimentation, Candida tropicalis (C. tropicalis, ATCC 62690) was employed as a representative example of yeasts to challenge the antifungal functions of the polymeric silver sulfadiazines. Briefly, C. tropicalis was grown in Yeast and Mold (YM) broth at 26° C. for 48 hours, harvested by centriftge, washed with sterile PBS, and resuspended in sterile PBS to densities of 10⁸-10⁹ CFU/mL. 10 μL of the C. tropicalis suspensions were placed between two identical polymeric silver sulfadiazine films (2×2 cm), and a sterile weight (100 g) was added onto the films. After a certain period of contact time, the films were transferred into 10 mL sterilize PBS, sonicated for 5 minutes, and then vortexed for 1 minute. An aliquot of the solution was serially diluted, and 100 μL of each dilution were plated onto YM agar plates. Colony-forming units on the agar plates were counted after incubation at 26° C. for 48 hours. Pure PMMA films and the correspondent ASD-MMA copolymer films without silver nitrate treatment were tested under the same conditions to serve as controls. Each test was repeated three times.

In investigating the structural stability of the samples, a series of polymeric silver sulfadiazine films (2×2 cm) were immersed in 100 mL deionized water under constant shaking at room temperature. After a certain period of time, 1 mL solution was taken out of the immersing water and tested with a Beckman DU® 520 UV/VIS spectrophotometer in the range of 190-400 nm to determine whether ASD-containing chemicals were released from the film into the solution (characteristic absorption peaks of pure ASD: 239 and 261 nm). Afterwards, the water sample was also tested with 0.1 M potassium iodide aqueous solution to check for color change in order to decide whether silver cations were presented in the soaking solutions.

The antimicrobial function of the polymeric silver sulfadiazines was also assessed by a modified Kirby-Bauer (KB) technique. In this study, the surface of a Luria-Bertant (LB) agar plate and tryptic soy agar plate was overlaid with 1 mL of approximately 10⁸ to 10⁹ CFU/mL of E. coli and S. aureus, respectively. The plates were then allowed to stand at 37° C. for 2 hours. polymeric silver sulfadiazines film (1×1 cm) was placed onto the surface of each of the bacteria-containing agar plates. The film was gently pressed with a sterile forceps to ensure full contact between the film and the agar. The same procedure was also applied to the pure PMMA films and the correspondent ASD-MMA copolymer films without silver nitrate treatment to serve as controls. After incubation at 37° C. for 24 hours, the inhibition zone around the films (if any) was measured. Afterwards, films were removed sterilely from the agar plates, washed gently with non-flowing PBS (3×10 mL) to remove loosely attached cells. The resultant films were sonicated for 5 minutes and vortexed for 1 minute in 10 mL PBS. The solution was serially diluted, and 100 μL of each dilution was plated onto the correspondent agar plates. Recoverable microbial colonies were counted after incubation at 37° C. for 24 hours.

The polymeric silver sulfadiazine films were tested for retention of antibacterial and antifungal functions under storage. Films with known bound silver contents were stored under normal lab conditions (25° C., 30-90% RH). The silver contents and the antibacterial and antifungal functions were tested periodically over a 12-month storage time.

The durability was also tested after simulated usage/recharge cycles. In this experiment the polymeric silver sulfadiazine films were first treated with saturated NaCl aqueous solution at room temperature for 24 hours to partially quench the bound silver, and then recharged with silver nitrate solutions using the same conditions of the original samples. After different cycles of this “quenching-recharging” treatment, the silver contents and antibacterial and antifungal functions of the resultant films were reevaluated.

### Coating Results

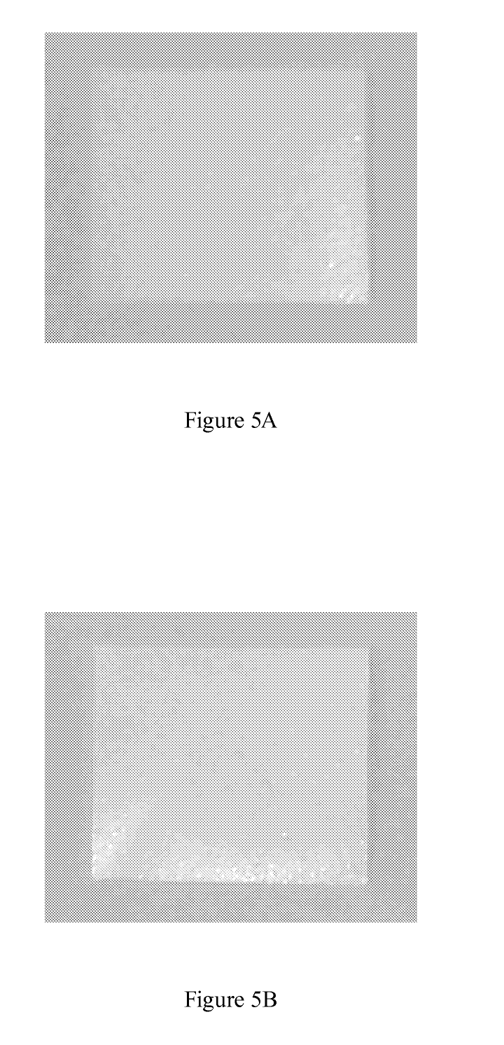

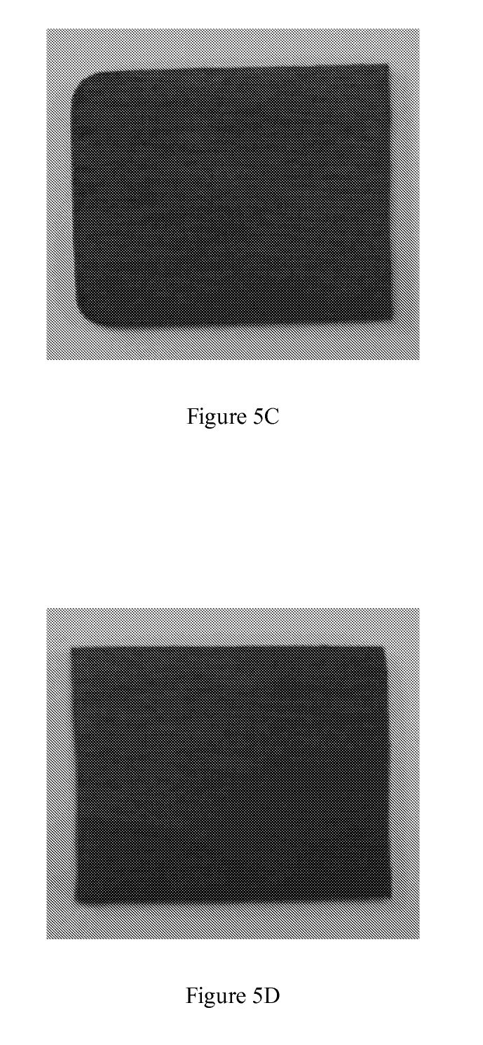

While the poly(Cl-TMPM) emulsion itself could be used as a paint-like coating to provide potent antimicrobial functions, the major focus of this study was to use the poly(Cl-TMPM) emulsion as an additive in commercial water-based latex paints (which are gaining increasing importance in the paint industry due to their “greener” nature than solvent-based paints) to transform the conventional paints into antimicrobial paints. It was encouraging to find that the poly(Cl-TMPM) emulsions could be freely mixed with most commercial water-based paints at any ratios without coagulation and/or phase separation. The covering capacity and appearance of the new paints were not negatively affected by the presence of poly(Cl-TMPM). As an example, FIG. 5 showed the same polystyrene plastic films painted with a commercial white paint and blue paint, and with the new paints containing 20 wt % (by solid content) of poly(Cl-TMPM), respectively.

The antimicrobial functions of the painted plastic films were tested by placing microbial suspensions on the paint surfaces for a certain period of time. Without the polymeric N-halamine emulsions, the commercial paints did not provide any antimicrobial functions after 1 hour of contact. After adding only 2% of the polymeric N-halamine emulsions into the same paints, the new paints provided a total kill of 107-108 CFU/mL of methicillin-resistant S. aureus (ATCC BAA-811), vancomycin-resistant E. faecium (ATCC 700221), E. coli (ATCC 15597), and C. Albicans (ATCC 10231) in 3 minutes, and 106-107 PFU/mL of MS-2 virus (ATCC 15597-B1) in 30 minutes. As a comparison, a commercially available MICROBAN®-based antimicrobial paint (DAP® Kwik Seal Plus®) was also tested under the same conditions, and that paint did not provide any inhibiting effect against any of the test species after up to 1 hour of contact.

The antimicrobial functions of the new paints are provided by the covalently bound chlorines of the polymeric N-halamines. The presence of covalently bound chlorines in the paints can be easily detected with potassium iodine/starch test strips (Fisher Scientific). As shown in FIG. 14, the test strip contacted with the original paint did not show any color change (FIG. 14A); the test strip contacted with the same paint containing 2% of the polymeric N-halamine emulsions, however, changed to dark blue within 1 min (FIG. 14B).

The covalently bound chlorines of the polymeric N-halamines in the paints are very stable. Iodometric titration showed that the chlorine content was not changed upon repeated touching with hands, wiping with cellulosic cleaning cloth saturated with soap and water, and even immersing in water for 2 weeks. Furthermore, after two weeks of immersing, iodometric titration and potassium iodine/starch test did not find any free chlorine in the immersing water, indicating that the polymeric N-halamine-based paints provided antimicrobial functions through contact kill, and the covalently bound chlorines did not leach out of the paint into the surrounding environment. In real applications, this non-leaching characteristic is expected to lead to long-lasting antimicrobial action of the new paints. Furthermore, the non-leaching property will also help to eliminate the concern of biocidal agents entering the surrounding environments to cause undesirable complications, making the new paints even more attractive for a wide range of applications.

To test the rechargeability of the covalently bound chlorines, polystyrene films painted with the new paint containing 2% of polymeric N-halamines were first immersed in 0.03% sodium thiosulfate aqueous solutions for 60 min to quench the chlorines, and then wiped for 1 minute with a 1:100 dilution of sodium hypochlorite bleach using a cellulosic cleaning cloth to recharge the chorines. The films were left air dry for 24 hours. After 3 cycles of this “quenching-recharging” treatment, the antimicrobial functions of the new paints were essentially unchanged.

Studies strongly indicate that polymeric N-halamine emulsions can be prepared by emulsion polymerization of N-halamine monomers. The polymeric N-halamine emulsions can be used as antimicrobial ingredients of conventional latex paints to provide potent antimicrobial functions against a wide range of microorganisms. The antimicrobial functions are stable, easily monitor-able, and rechargeable.

The antibacterial, antifungal, and antiviral efficacies of the poly(Cl-TMPM)-containing paints were evaluated, as discussed above, under both waterborne and airborne test conditions. The original commercial paints were used as controls, which did not show any antimicrobial effects. The poly(Cl-TMPM)-containing paints, however demonstrated encouraging antimicrobial efficacy, as summarized in the table below:

| Antimicrobial | | S. aureus | E. coli | MRSA | VRE | C. tropicalis | MS2 |
|---|---|---|---|---|---|---|---|
| test method | Content (%) | (min) | (min) | (min) | (min) | (min) | (min) |
| Waterborne | 1 | 120 | 60 | 60 | 120 | 120 | |
| Waterborne | 2 | 60 | 30 | 30 | 60 | 60 | |
| Waterborne | 5 | 10 | 5 | 10 | 30 | 30 | 240 |
| Waterborne | 10 | 5 | 5 | 5 | 10 | 30 | 120 |
| Waterborne | 20 | 2 | 2 | 2 | 5 | 10 | 60 |
| Airborne | 5 | 30 | 30 | 30 | 30 | 60 | |
| Airborne | 10 | 30 | 10 | 10 | 10 | 30 | |

*S. aureus, E. coli, MRSA, VRE, C. tropicalis concentrations were 10⁸-10⁹ CFU/mL, and MS2 virus density was 10⁸-10⁹ PFU/mL; the new paints contained 1-20 wt % poly(Cl-TMPM). Each test was repeated three times, and the longest minimum contact time for a total kill of the microbes (the weakest antimicrobial efficacy observed) was reported.

In waterborne tests, poly(Cl-TMPM) contents showed a significant influence on antimicrobial potency. For example, with 1 wt % of poly(Cl-TMPM), it took the paints 120 min and 60 min to provide a total kill of 10⁸-10⁹ CFU/mL of S. aureus 6538 (Gram-positive bacteria) and E. coli 15597 (Gram-negative bacteria), respectively. When the poly(Cl-TMPM) content was increased to 5 wt %, the contact time for a total kill of the same species dramatically decreased to 10 min and 5 min, respectively.

It was a striking finding that the poly(Cl-TMPM)-containing paints provided potent antibacterial activity against drug-resistant species including MRSA BAA-811 and VRE 700221, which are major concerns in healthcare settings and a wide range of related community facilities, causing serious healthcare-related infections and community acquired infections. These results pointed to great potentials of the new poly(Cl-TMPM)-containing paints for use in antimicrobial surfacing in related facilities to help reduce the risk of such infections.

The antifungal function of the new paints was evaluated with C. tropicalis 62690, and at 5 wt % of poly(Cl-TMPM) content, the new paints provided a total kill of 10⁸-10⁹ CFU/mL of the yeast in 30 min in waterborne tests. Higher poly(Cl-TMPM) contents led to even faster antifungal action. The virus (E. coli bacteriophage MS2), which has been widely used as surrogate of enteric viral pathogens, was relatively difficult to kill. With 5% of poly(Cl-TMPM), it took 240 min for the new paint films to offer a total kill of 10⁸-10⁹ PFU/mL of the virus in the waterborne test. When the content of poly(Cl-TMPM) was increased to 10 wt % and 20 wt %, the contact time for a total kill of the virus decreased to 120 min and 60 min, respectively.

The airborne antimicrobial efficacies of the poly(Cl-TMPM)-containing paint films were challenged with S. aureus 6538, E. coli 15597, MRSA BAA-811, VRE 700221, and C. tropicalis 62690. To simulate the deposition of airborne microorganisms, a common route of spreading infectious agents generated, for example, by talking, sneezing, coughing, or just breathing, a small commercial sprayer was used to spray the test organisms onto the poly (Cl-TMPM)-containing paint films. The table above provides results. It was found that at the same poly(Cl-TMPM) content, the contact time for a total kill of the same species was slightly longer under the airborne conditions than that in the waterborne conditions. This could be caused by the antimicrobial mechanism of N-halamines. It has been suggested that N-halamines provided antimicrobial effects by donating chlorines to microbial cells, leading to expiration of the microorganisms. Under airborne conditions, less water/moisture was involved when the microbial aerosols made contact with the paints, thus, a longer contact time was needed for a total kill. Nevertheless, even under the airborne conditions, the new paints could still provide a total kill of 10⁸-10⁹ CFU/mL of the bacteria (including the drug-resistant species) and yeast in 30-60 min at 5 wt % of poly(Cl-TMPM) content. When the poly(Cl-TMPM) content was increased to 10 wt %, the contact time for at total kill of the bacteria or the yeast was further reduced to 10-30 min.

In addition to antibacterial (including the drug-resistant species), antifungal, and antiviral functions, the new poly(Cl-TMPM)-containing paints demonstrated potent anti-mold function. As shown in the table below, after 1 month of growth, about 30% of the original paint surface was already covered by mold.

| | Surface mold covering ratio (%) | | |
|---|---|---|---|
| | 0 wt % | 5 wt % | 10 wt % |
| Time | poly(Cl- | poly(Cl- | poly(Cl- |
| (month) | TMPM) | TMPM) | TMPM) |
| 0.5 | <10 | 0 | 0 |
| 1 | 30 | 0 | 0 |
| 2 | 60 | 0 | 0 |
| 3 | 100 | 0 | 0 |

When the growing time was extended to 3 months, 100% of the original paint surface was covered by mold. On the new paints containing 5 wt % or 10 wt % of poly(Cl-TMPM), however, no any mold growth could be detected during the 3-month testing period. As the general public are increasingly concerned about mold growth and indoor mold exposure, the anti-mold effects of the poly(Cl-TMPM)-containing paints would further strengthen the potential for the new paints to be used in real applications.

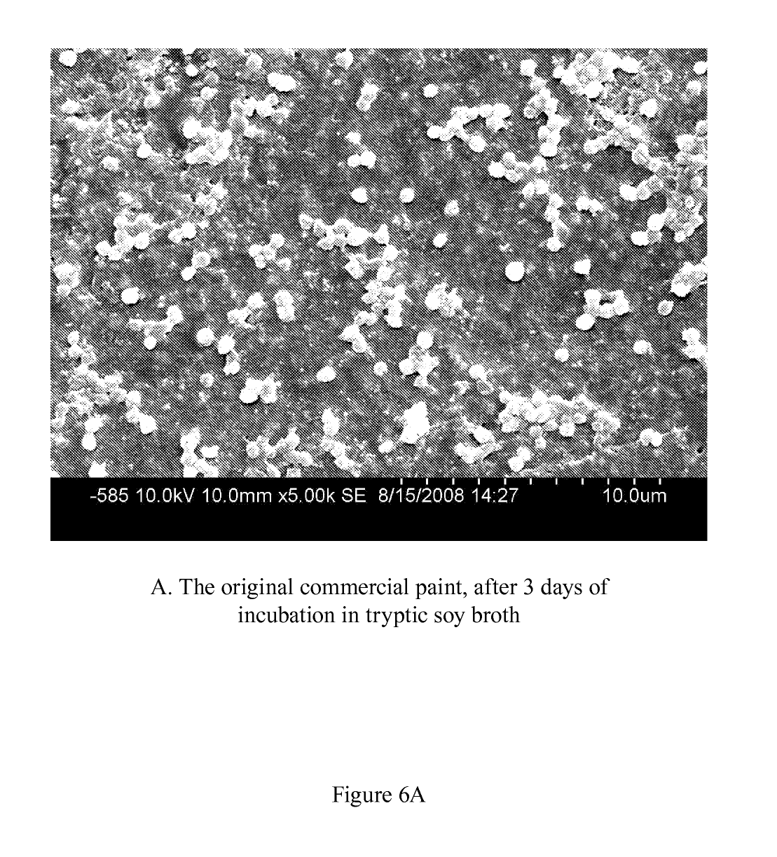

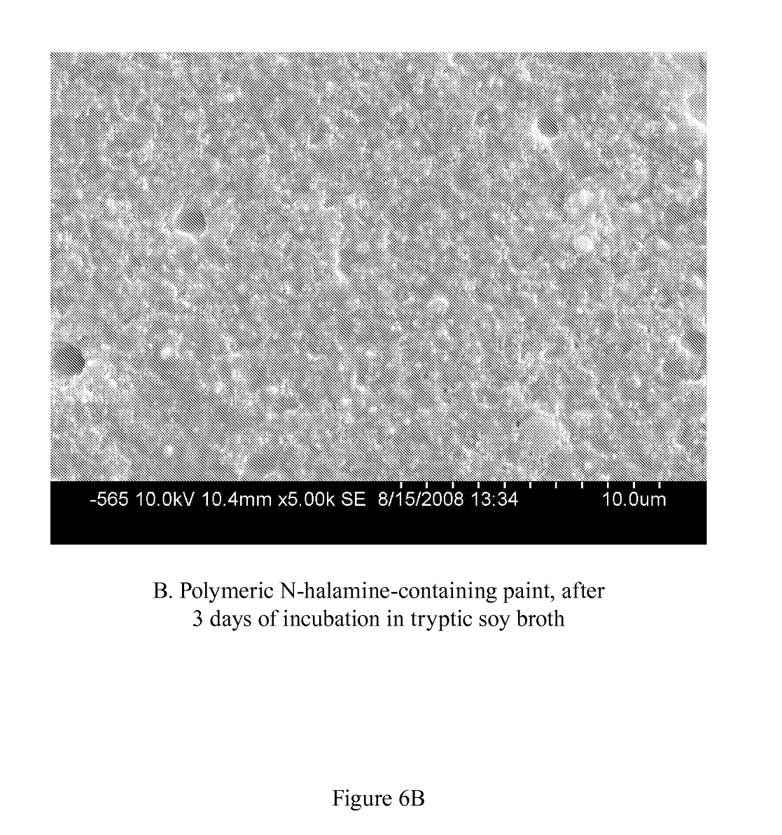

The formation and development of biofilms can cause serious industrial, environmental and institutional problems. To provide detailed information about the biofilm-controlling effect, the original paint film and the new paint film containing 10 wt % of poly(Cl-TMPM) were contacted with S. aureus 6538 for 30 min to allow initial adhesion, and the samples were then immersed in tryptic soy broth to facilitate formation and development of bacterial biofilms. As shown in FIG. 6, after 3 days of incubation, a large amount of bacteria adhered onto the surface of the original commercial paint film, forming micro-colonies and developing into biofilms (FIG. 6 A). On the other hand, the poly(Cl-TMPM)-containing paint film showed a much clearer surface (FIG. 6 B): no adherent bacteria could be observed, and no biofilms were formed, suggesting potent biofilm-controlling activity.

To provide a deeper understanding about the antimicrobial action of the poly(Cl-TMPM)-containing paints, zone of inhibition studies of the samples were performed. As shown in the table below, the original commercial paint did not provide any inhibition zones against S. aureus 6538 or E. coli 15597.

| | Inhibition zone | | Bacteria recovered | |
|---|---|---|---|---|
| Poly(Cl-TMPM) | (mm) | | (CFU/cm²) | |
| content | S. aureus | E. coli | S. aureus | E. coli |
| 0 | 0 | 0 | 4.7 × 10⁶ (±1.7 × 10⁵) | 1.9 × 10⁶ (±1.6 × 10⁵) |
| 5 wt % | 1.9 ± 0.1 | 2.2 ± 0.1 | 1.5 × 10³ (±2.8 × 10²) | 7.2 × 10² (±6.4 × 10¹) |
| 10 wt % | 2.3 ± 0.2 | 2.4 ± 0.1 | 5.0 × 10¹ (±3.7 × 10⁰) | 1.6 × 10¹ (±7.2 × 10⁰) |

However, the new paints containing 5 wt % of poly(Cl-TMPM) generated a zone of 1.9±0.1 mm against S. aureus 6538, and a zone of 2.2±0.1 mm against E. coli 15597 (n=3). Further increasing poly(Cl-TMPM) content to 10 wt % did not significantly increase the zone sizes against the Gram-positive or the Gram-negative bacteria.

After zone of inhibition tests, the paint film samples were washed and sonicated to recover surface adherent bacteria. As shown in the table, from the original commercial paint film, as high as 4.7×10⁶ (±1.7×10⁵) CFU/cm² of S. aureus 6538 or 1.9×10⁶ (±1.6×10⁵) CFU/cm² of E. coli 15597 could be recovered (n=3). From the paint films containing 5 wt % of poly(Cl-TMPM), the recoverable level of S. aureus 6538 decreased to 10³ CFU/cm², and the recoverable level of E. coli 15597 dropped to 10² CFU/cm². When the poly(Cl-TMPM) content was increased to 10 wt %, the levels of the recoverable bacteria further decreased to the range of 10¹ CFU/cm².

These results suggested that during the tests, at least some of the antimicrobial agents diffused away from the poly(Cl-TMPM)-containing paint films to kill the bacteria. To determine what are responsible for this action, a series of the new paint films containing 10 wt % of poly(Cl-TMPM) (2×2 cm) were immersed in 10 mL deionized water under constant shaking at room temperature, and an UV/VIS spectrophotometer was used to test the immersing solutions. Within the test period of 72 h, the soaking solution was very clear, and no suspensions/precipitation was observed. In the range of 190-400 nm, no UV absorption could be detected, suggesting that almost no detectable Cl-TMPM-containing compounds were released into the water system.

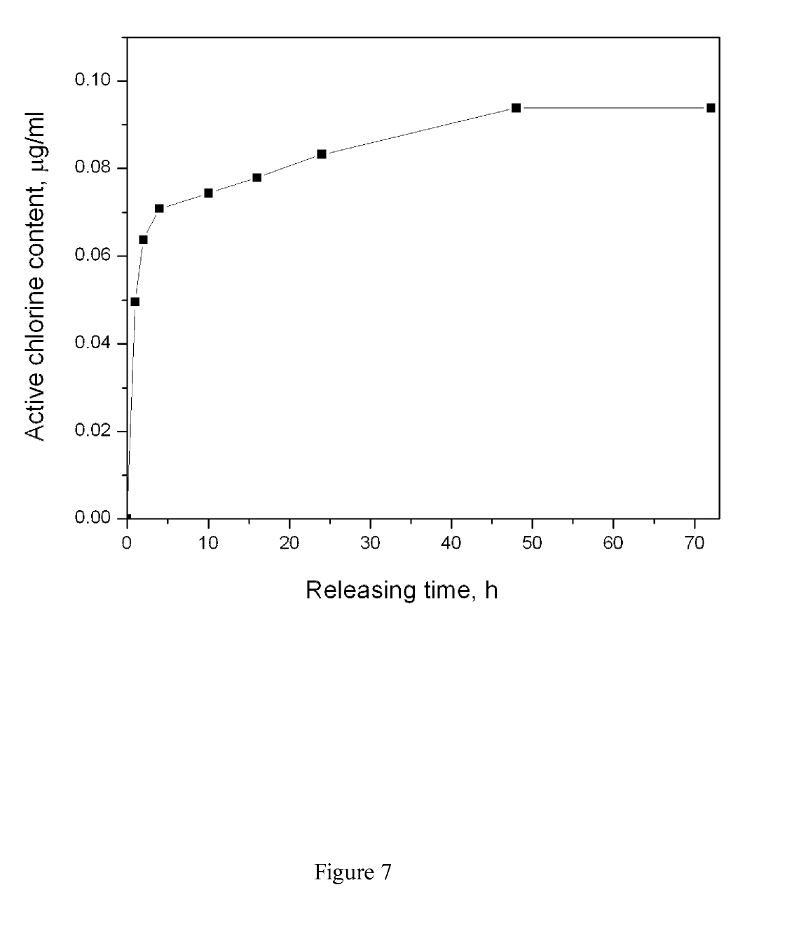

Thus, the inhibition zones could be created by positive chlorines generated by the disassociation of the amine N—Cl bonds. To confirm this, a quantitative evaluation of the positive chlorine contents in the immersing solutions was conducted by iodometric titration. FIG. 7 presented the positive chlorine content in the solution as a function of releasing time. It was found that in the initial stage (1 h to 4 h), the positive chlorine content gradually increased; after that, the increasing trend became much slower, and when the equilibrium of the dissociation of the N—Cl bond was achieved, the chlorine content in the solution was kept constant at around 0.094 μg/ml (0.094 ppm). This value is much lower than the current EPA Maximum Residual Disinfectant Level (MRDL) in drinking water of 4 ppm. In other words, although the new paints contained 10 wt % of poly(Cl-TMPM; 1.307% of covalently bound chlorines), only 0.094 μg/mL of positive chlorine would be released from the paint films under equilibrium conditions if no microbial challenges were presented.

On the other hand, in the presence of microbial challenges (as seen in the Zone of inhibition study and antimicrobial tests), the disassociated chlorines could be quickly consumed by the surrounding microorganisms. This disturbed the N-halamine disassociation equilibrium, resulting in more chlorine to be continuously released to maintain the equilibrium. Thus, an inhibition zone and relatively rapid antimicrobial action could be observed. After all the microbial challenges were cleared, however, the N-halamine disassociation equilibrium could be easily achieved and maintained, thus, a very small amount of dissociated chlorines would be presented (0.094 ppm under our testing conditions), and this would lead to exceptional chlorine storage stability.

The non-leaching nature of the Cl-TMPM-containing components in the paints and the extremely low level of disassociation of the amine N—Cl bonds led to excellent durability of the new poly(Cl-TMPM)-containing paints. Under normal lab conditions (25° C., 30-90% RH), the paint samples have been stored for more than 12 months without any significant changes of the active chlorine contents in the paints as well as the antimicrobial efficacies against the bacteria and yeast species, pointing to long antimicrobial durations in real applications.

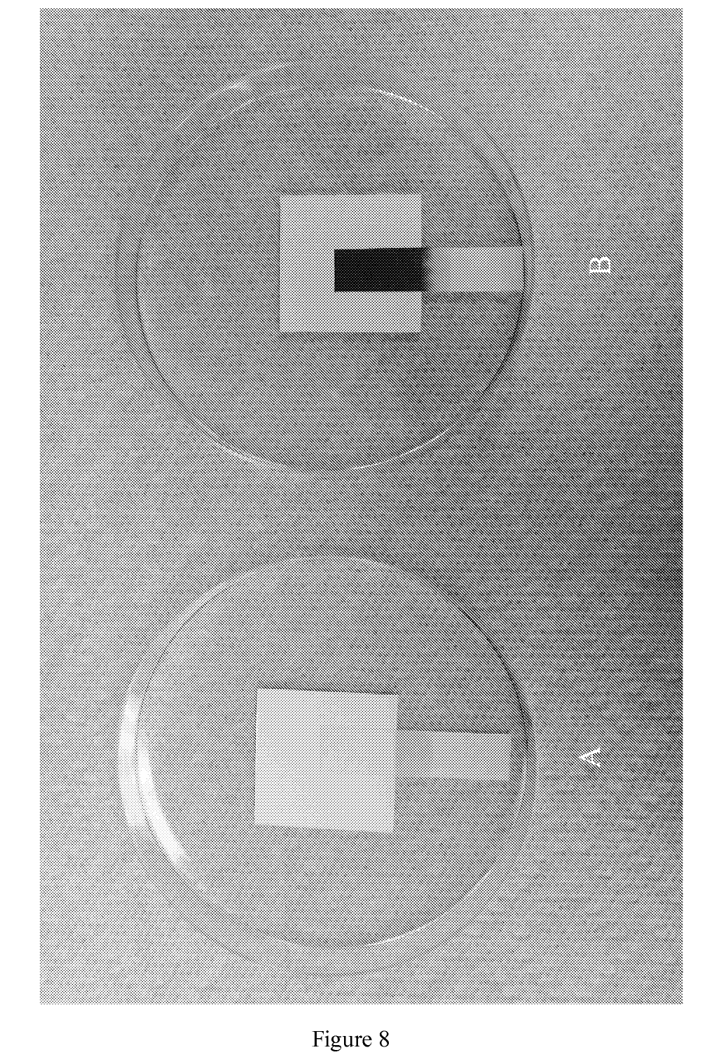

On the other hand, challenging conditions (e.g., heavy soil, flooding, etc.) in real applications might consume more chlorine and thus shorten the antimicrobial duration. Nevertheless, the antimicrobial function of the new poly(Cl-TMPM)-containing paints can be easily monitored with a simple potassium iodine/starch test by contacting the paint surface with potassium iodine/starch test strips on an unobvious spot. As shown in FIG. 8 as a demonstration, poly(Cl-TMPM) in the new paints would react with potassium iodine to produce iodine, and this would generate a dark blue color with starch almost instantly. This simple test could be performed even by the end users in real applications, and if potassium iodine test showed that the antimicrobial function was lost, the lost chlorines could be recharged by another chlorination treatment.

To preliminarily evaluate rechargeability, a series of the new paint films containing 5 wt % of poly(Cl-TMPM) were first treated with 0.3% sodium thiosulfate to quench the active chlorine and then rebleached with 1% of DCCNa at room temperature (see the Experimental section for details). After 10 cycles of the quenching-rebleaching treatments, the chlorine contents and antimicrobial activities of the new paints were essentially unchanged, indicating that the antimicrobial function was fully rechargeable.

Polymeric N-halamines prepared from monomers containing at least one kind of monomer selected from Formulas 2-16 showed similarly powerful, durable and renewable antimicrobial/biocidal properties.

### Testing Grafted Fabrics

In testing the antibacterial activities of the PTMPAM-grafted-fabrics, the antibacterial functions of the chlorinated PTMPMA-g-fabrics were challenged with 10⁶-10⁷ CFU/mL of S. aureus (ATCC 6538, gram-positive), S. epidermidis (ATCC 35984, gram-positive) and E. coli (ATCC 15597, gram-negative). The results are summarized in the Table below:

ANTIBACTERIAL ACTIVITIES OF CHLORINATED
PTMPMA-G-FABRICS

| Active chlorine | Minimum contact time | | |
|---|---|---|---|
| content of fabrics | for total kill (minutes) | | |
| (percent) | S. aureus | S. epidermidis | E. coli |
| 0.45 | 30 | — | 30 |
| 0.78 | — | 30 | 30 |
| 1.55 | 20 | — | 30 |
| 2.56 | — | 20 | 20 |

In testing the antibacterial activities of the Cl-TMPM-grafted-fabrics, the antibacterial functions of the chlorinated PTMPMA-g-fabrics were challenged with 106-107 CFU/mL of S. aureus (ATCC 6538, gram-positive), S. epidermidis (ATCC 35984, gram-positive) and E. coli (ATCC 15597, gram-negative) with chlorine contents of 0.5%, 0.9%, and 1.8%. All the samples tested provided a total kill of 106-107 CFU/mL of the test species within 30 minutes. Active chlorine content of the samples did not seem to significantly affect the antimicrobial potency. Other previous studies showed that if cotton fabrics were grafted with amide-based N-halamines, with less than 1% of active chlorine content, the fabrics provided a total kill of 108-109 CFU/mL of E. coli and S. aureus in only 3 minutes. Since the bactericidal action of N-halamines is believed to be caused by the transfer of positive halogens from the N-halamines to appropriate receptors in the bacteria cells, these findings imply that the piperidyl-based amine N-halamines in the Cl-TMPM-g-fabric are very stable.

The most significant result is that all of the samples tested provided a total kill of 10⁶-10⁷ CFU/mL of the test species within 30 minutes. Active chlorine content of the samples did not seem to significantly affect the antimicrobial potency. For example, with 0.45% of active chlorine, the fabric provides a total kill of S. aureus and E. coli in 30 minutes. When the active chlorine content is increased to 1.55%, it still takes the sample 30 minutes to kill 10⁶-10⁷ CFU/mL of E. coli, and 20 minutes to kill the same amount of S. aureus. Other previous studies showed that if cotton fabrics were grafted with amide-based N-halamines, with less than 1% of active chlorine content, the fabrics provided a total kill of 10⁸-10⁹ CFU/mL of E. coli and S. aureus in only 3 minutes. Since the bactericidal action of N-halamines is believed to be caused by the transfer of positive halogens from the N-halamines to appropriate receptors in the bacteria cells, these findings imply that the piperidyl-based amine N-halamines in the PTMPMA-g-fabric are very stable.

In testing the stability, durability, and rechargeability of the active chlorines and antimicrobial activities of the PTMPMA-grafted-fabrics, the hydrolytic and thermal stability of the N—Cl bonds in the chlorinated PTMPMA-grafted-fabrics was first challenged with autoclave treatment in a pressure steam sterilizer at 124-126° C. for 15 minutes, according to the autoclave manufacturer's recommendation for sterilization. After this treatment, 89.5%, 87.1% and 77.8% of the original active chlorines were retained in the chlorinated fabrics with 17.8%, 10.8% and 2.7% of graft yield, respectively, and the antimicrobial activities of the autoclaved samples were essentially unchanged. Each titration was performed five times. This is summarized in the Table below.

ACTIVE CHLORINE CONTENT AFTER TREATMENTS

| | active chlorine content at different | | |
|---|---|---|---|
| | graft yield (weight percent) | | |
| Samples | 17.8 | 10.8 | 2.7 |
| freshly chlorinated | 2.56 ± 0.03 | 1.55 ± 0.01 | 0.45 ± 0.02 |
| after steam sterilization | 2.29 ± 0.10 | 1.35 ± 0.01 | 0.35 ± 0.02 |
| after 30 rounds laundry | 2.32 ± 0.02 | 1.45 ± 0.03 | 0.32 ± 0.01 |
| after 10 rounds recharge | 2.41 ± 0.04 | 1.47 ± 0.05 | 0.44 ± 0.03 |

In testing the stability, durability, and rechargeability of the active chlorines and antimicrobial activities of the Cl-TMPM-grafted-fabrics, the hydrolytic and thermal stability of the N—Cl bonds in the chlorinated Cl-TMPM-grafted-fabrics was first challenged with autoclave treatment in a pressure steam sterilizer at 124-126° C. for 15 minutes, according to the autoclave manufacturer's recommendation for sterilization. After this treatment, >75% of the original chlorine was retained, and the antimicrobial activities of the autoclaved samples were essentially unchanged.

Since a wide range of medical/hospital articles are required to be sterilized before they can be used, and autoclave is still the most widely used sterilization method in general practice, these findings point to significant potentials of the new amine N-halamine-based fibrous materials.

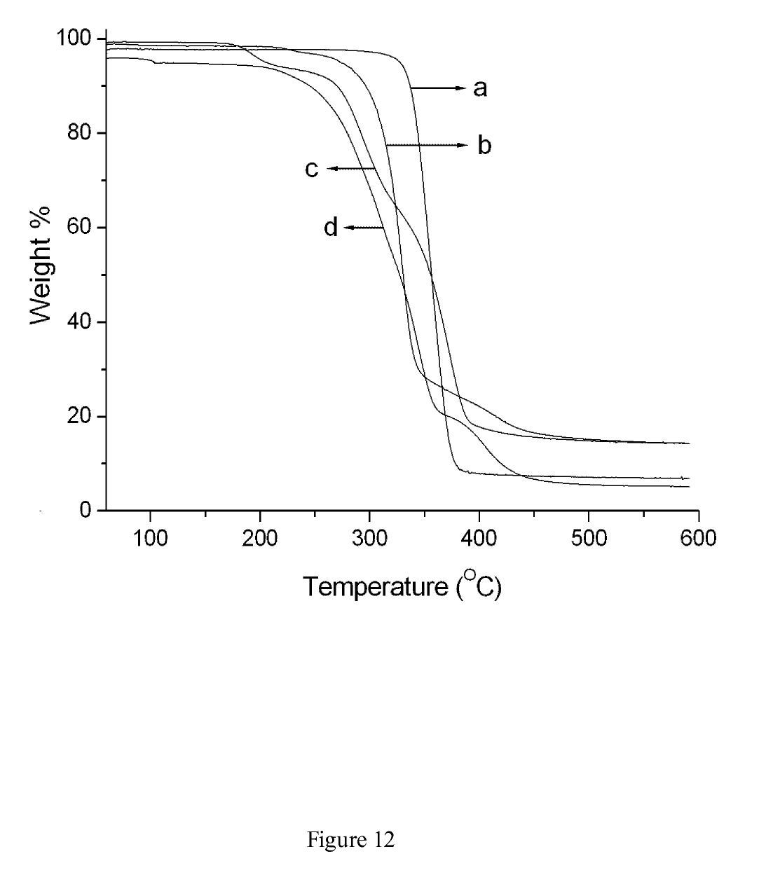

The thermal stability of the N—Cl bond in chlorinated PTMPMA-grafted-fabrics was investigated with thermogravimetric analysis (TGA). As shown in FIG. 12, pure cotton fabric does not show any significant weight loss before 300° C. (FIG. 12a). Both pure PTMPMA (FIG. 12d) and PTMPMA-grafted-fabrics (graft yield: 17.8%, FIG. 12b) begin to lose weight starting from around 230° C., which corresponds to the thermal decomposition of PTMPMA polymer chain. In the TGA curve of the chlorinated PTMPMA-grafted-fabric, the sample displays noticeable weight loss starting from 180° C. (FIG. 12c), and this is most likely caused by the thermal decomposition of the samples which was induced/accelerated by the N—Cl bond breakage. Given the fact that the autoclave treatment was conducted at 124-126° C., these TGA results strongly suggest that the N—Cl bonds in chlorinated PTMPMA-grafted-fabrics are thermally stable enough to survive autoclaves.

Durability and rechargeability are two other important features of the new hindered amine N-halamine-based fibrous materials. At 20-25° C. and 30-90% RH, the samples have been stored for more than 10 months without any significant changes of the active chlorine contents on the fabrics as well as the antimicrobial efficacies against E. coli and S. aureus. In machine washing test, even after 30 rounds of continuous washing without chlorination treatment, the samples still retained at least 71% of the original active chlorines, further confirming the hydrolytic stability of the N—Cl bonds.

To test rechargeability, the Cl-TMPM-grafted fabrics and the chlorinated PTMPMA-grafted-fabrics were first treated with 0.3% of sodium thiosulfate solution to partially quench the active chlorine for 1 h, and then rechlorinated with 0.1% of sodium hypochlorite solution at room temperature for 30 minutes. After 10 cycles of the quenching-rechlorinating treatment, at least 94% of the original active chlorine was retained, and the antimicrobial activities were unchanged.

Therefore, the polymerizable hindered amine monomers, (TMPMA) and Cl-TMPM, were successfully grafted onto cotton cellulose via free radical polymerization with the initiation of Ceric salt. The grafted fabrics were treated with diluted sodium hypochlorite solution to transform the N—H bond in the grafted TMPMA chains into amine N-halamines. The new polymeric N-halamine fibrous materials demonstrate powerful, durable, and rechargeable antibacterial activities against both gram-positive and gram-negative bacteria. Thanks to the excellent hydrolytic stability and thermal stability, the active chlorines in the new polymeric N-halamine fibrous materials are autoclavable without significantly degrading the desirable characteristics of the materials, making the new materials attractive candidates for a wide range of applications.

### Silver Sulfadiazine Results

Both pure poly (methyl methacrylate (PMMA) and ASD-MMA copolymer (without silver nitrate treatments) films were used as controls. Pure PMMA did not provide any inhibiting effects against the test organisms within the test period up to 2 hours. Moreover, while SD is a potent antibiotic that has been successfully used to treat urinary tract infections and combined with pyrimethamine to treat toxoplasmosis, without the silver nitrate treatment, the ASD-MMA copolymer did not show any noticeable antibacterial or antifungal activities under the test conditions. Since SD is believed to eliminate bacteria by stopping the production of folic acid inside the bacterial cell, this finding indicates that (1) the size of the ASD-MMA copolymer is too big to penetrate into the microbial cells; and (2) during the antimicrobial tests, no monomeric structures containing SD moieties leached out of the ASD-MMA copolymer films to provide antimicrobial function, suggesting that the ASD-MMA copolymer structure is relatively stable.

In contrast, after silver nitrate treatment, the ASD-MMA copolymer was transformed into polymeric silver sulfadiazine, and this transformation led to potent biocidal activities in the product. At 1.29% surface bound silver content, the polymeric silver sulfadiazine provided a total kill of approximately 10⁸ to 10⁹ CFU/mL of E. coli and S. aureus in a period of 10 minutes, and a total kill of approximately 10⁸ to 10⁹ CFU/mL of C. tropicalis in a period of 30 minutes. This data is outlined in the Table below:

Percentage reduction of S. aureus, E. coli, and C. tropicalis (%)*

| Contact time | | | |
|---|---|---|---|
| (min) | S. aureus | E. coli | C. tropicalis |
| 5 | 99.9 | 99.9 | 90 |
| 10 | Total kill | Total kill | 99 |
| 30 | Total kill | Total kill | Total kill |

*S. aureus, E. coli, and C. tropicalis concentrations were 10⁸-10⁹ CFU/mL; the polymeric silver sulfadiazine contained 1.29% surface bound silver based on XPS study.

In parallel to the antibacterial and antifungal studies conducted, a series of the polymeric silver sulfadiazine films (2×2 cm) were immersed in 100 mL deionized water under constant shaking at room temperature, and an UV/VIS spectrophotometer was used to test the immersing solutions. Within the test period of 24 hours, in the range of approximately 190 to approximately 400 nm, no UV absorption was detected. Moreover, potassium iodine test did not show any color change of the immersing solutions. These results suggest that no detectable monomeric SD/ASD components or silver cations were released into the surrounding environment under the testing conditions, indicating that the polymeric silver sulfadiazines might provide biocidal functions primarily through direct contact.

A zone of inhibition test was performed to provide further information about any “contact kill” mechanism of action, and showed that neither pure PMMA and ASD-MMA copolymer nor the polymeric silver sulfadiazine films provided any inhibiting zone during the test period of 24 hours. After zone of inhibition test, the film samples were washed and sonicated to recover surface adherent bacteria. From pure PMMA film surfaces, (3.95±0.64)×10⁴ CFU/cm² of S. aureus or (7.24±0.42)×10⁴ CFU/cm² of E. coli were recovered (n=3). From ASD-MMA film surfaces, (3.90±0.14)×10⁴ CFU/cm² of S. aureus or (6.85±0.94)×10⁴ CFU/cm² of E. coli were recovered. On polymeric silver sulfadiazine films, however, the numbers of the recoverable bacteria were only in the range of 10⁰ CFU/cm². This data is summarized in the Table below:

| | | ASD-MMA | Polymeric silver |
|---|---|---|---|
| Bacteria | PMMA film | copolymer film | sulfadiazine film** |
| S. | (3.95 ± 0.64) × 10⁴ | (3.90 ± 0.14) × 10⁴ | (7.33 ± 1.52) × 10⁰ |
| aureus | | | |
| E. coli | (7.24 ± 0.42) × 10⁴ | (6.85 ± 0.94) × 10⁴ | (3.00 ± 0.57) × 10⁰ |

The findings establish that the polymeric silver sulfadiazine samples kill microbes mainly by direct contact. During the test, no zone of inhibition was observed, indicating that almost no monomeric antimicrobial agents (e.g., SD/ASD moiety or silver cations) leached out of the film samples. Only the microbes that made contact with the polymeric silver sulfadiazine samples were killed, and the surrounding cells were not affected. In real applications, this non-leaching characteristic may provide a number of advantages. The most obvious advantage is improved durability of the antimicrobial effects. Because almost none of the antimicrobial agents (i.e., silver cations) are released and thus consumed by surrounding cells, polymeric silver sulfadiazine samples may provide long-term protections against microbial adhesion. Further, the non-leaching property may help to eliminate concern regarding antimicrobial agents entering the surrounding environments to cause undesirable complications, making the polymeric silver sulfadiazines attractive candidates for a number of potential biomedical applications.

The biocidal functions of the polymeric silver sulfadiazines were both durable and rechargeable. At 21° C. and 30-90% RH, the samples were stored for more than 12 months without any significant changes in the silver content on the films as well as no significant changes in the biocidal efficacies against the bacterial and fungal species. Films containing 1.29% of surface bound silver have also been treated with saturated NaCl aqueous solution for 24 hours to partially quench the active silver, and then re-treated with 0.01 M AgNO₃ aqueous solution to recharge the consumed silver. After 10 cycles of the “quenching-charging” treatments, the silver contents and biocidal activities of the samples were essentially unchanged, indicating that the antibacterial and antifungal functions were fully rechargeable. The C-SD treated polymeric silver sulfadiazines showed similar antimicrobial performance.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

The following is claimed:

1. An antimicrobial composition comprising:
a renewable antimicrobial material;
wherein the renewable antimicrobial material includes a consumable portion that can be replenished after it is consumed.

2. The antimicrobial composition of claim 1, wherein the renewable antimicrobial material comprises an N-halamine derivative.

3. The antimicrobial composition of claim 1, wherein the renewable antimicrobial material comprises one or more monomers of Formula I or Formula II 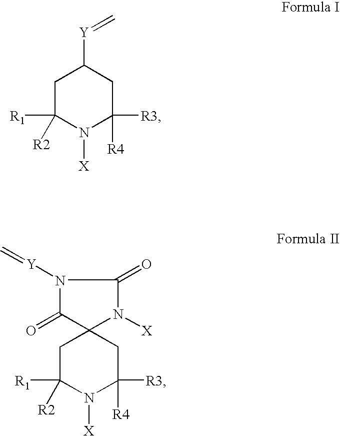
in which R1, R2, R3, R4, and Y are C₁ to C₄₀ alkyl, C₁ to C₄₀ alkylene, C₁ to C₄₀ alkenyl, C₁ to C₄₀ alkynyl, C₁ to C₄₀ aryl, C₁ to C₃₀ alkoxy, C₁ to C₄₀ alkylcarbonyl, C₁ to C₄₀ alkylcarboxyl, C₁ to C₄₀ amido, C₁ to C₄₀ carboxyl, or combinations thereof, X is Cl, Br or H, and Z is Cl or Br.

4. The antimicrobial composition of claim 1, wherein the renewable antimicrobial material comprises one or monomers of Formula III, Formula IV, Formula V or Formula VI, respectively:

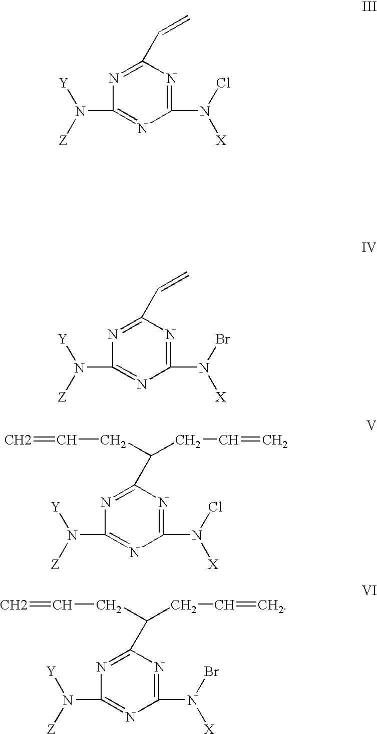

5. The antimicrobial composition of claim 1, wherein the renewable antimicrobial material comprises one or more of N-chloro-2,2,6,6-tetramethyl-4-piperidyl methacrylate, N-bromo-2,2,6,6-tetramethyl-4-piperidyl methacrylate, N-chloro-2,2,6,6-tetramethyl-4-piperidyl acrylate, or N-bromo-2,2,6,6-tetramethyl-4-piperidyl acrylate.

6. The antimicrobial composition of claim 4, wherein the renewable antimicrobial material comprises a polymer prepared by polymerizing or co-polymerizing one or more monomers of Formula I, Formula II, Formula III, Formula IV, Formula V or Formula VI.

7. The antimicrobial composition of claim 1, wherein the renewable antimicrobial material comprises poly (N-chloro-2,2,6,6-tetramethyl-4-piperdyl methacrylate).

8. The antimicrobial composition of claim 1, comprising a latex paint.

9. A method of forming an antimicrobial composition, the method comprising:
halogenating an N-halamine monomer; and
polymerizing the halogenated N-halamine derivative.

10. A method of forming an antimicrobial composition, the method comprising:
polymerizing an N-halamine monomer; and
halogenating the resulting polymer in subsequent applications to replenish depleted halogen ions.

11. A renewable antimicrobial film formed by coating and drying a solution of the renewable antimicrobial composition of claim 1.

12. A method of forming a renewable antimicrobial surface, the method comprising:
coating the renewable antimicrobial composition of claim 1; and
drying the coating.

13. The method of claim 12, further comprising subjecting the renewable surface to microbes and then renewing the renewable antimicrobial surface.

14. An antimicrobial polymeric material comprising:
a matrix; and
a renewable antimicrobial material bound to the matrix.

15. The antimicrobial fabric of claim 14, wherein the matrix comprises a fibrous material.

16. The antimicrobial fabric of claim 14, wherein the renewable antimicrobial material comprises N-halo-2,2,6,6-tetramethyl-4-piperdyl methacrylate.

17. The antimicrobial fabric of claim 14, wherein the renewable antimicrobial material comprises monomers selected from Formula I, Formula II, Formula III, Formula IV, Formula V or Formula VI.

18. The antimicrobial fabric of claim 17, wherein at least some halogen ions are consumed upon exposure to microbes, and the halogen ions can be replaced via a halogenation treatment.

19. An antimicrobial composition comprising:
a polymer or copolymer containing covalently bound sulfadiazine; and
silver cations bound to the sulfadiazine.

20. The antimicrobial composition of claim 19, comprising 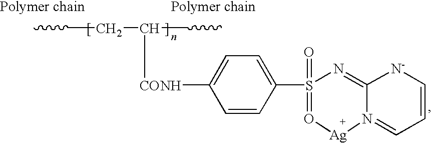
where the polymer chains can be any polymer and n is equal to or greater than 1.

21. The antimicrobial composition of claim 19, formed by reacting 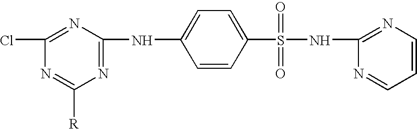
with a reactive site on a substrate, followed by contacting with silver nitrate,
where R can be Cl, C1 to C40 alkyl, C1 to C40 alkylene, C1 to C40 alkenyl, C1 to C40 alkynyl, C1 to C40 aryl, C1 to C30 alkoxy, C1 to C40 alkylcarbonyl, C1 to C40 alkylcarboxyl, C1 to C40 amido, C1 to C40 carboxyl, or combinations thereof.

22. The antimicrobial composition of claim 21, wherein the reactive site comprises one or more of —OH, —NH₂ or —SH.

23. A renewable antimicrobial material formed by coating and drying, mixing, blending, spraying or extruding a solution of the antimicrobial composition of claim 17.

24. The renewable antimicrobial material of claim 23, wherein exposure to microbes consumes at least some of the silver ions.

25. The renewable antimicrobial material of claim 24, wherein the silver ions can be replaced by exposing the antimicrobial material to a source of silver cation.

26. The renewable antimicrobial material of claim 25, wherein the source of silver cation comprises silver nitrate.
